(19) United States
(12) Patent Application Publication
(10) Pub. No.: US 2023/0346399 A1
(43) Pub. Date: Nov. 2, 2023

(54) DEVICES AND METHODS FOR REMOVING MATERIAL FROM A PATIENT

(71) Applicant: XTRACT MEDICAL, INC., Louisville, CO (US)

(72) Inventors: MICHAEL SCHALLER, Louisville, CO (US); JORGE GAMBA, JACKSONVILLE, FL (US)

(73) Assignee: XTRACT MEDICAL, INC., Louisville, CO (US)

(21) Appl. No.: 18/217,493

(22) Filed: Jun. 30, 2023

Related U.S. Application Data
(63) Continuation of application No. PCT/US2023/063580, filed on Mar. 2, 2023.
(60) Provisional application No. 63/415,201, filed on Oct. 11, 2022.
(60) Provisional application No. 63/359,990, filed on Jul. 11, 2022.
(60) Provisional application No. 63/315,764, filed on Mar. 2, 2022.

(51) Int. Cl. A61B 17/22; A61M 1/00

(52) U.S. Cl. CPC A61B 17/22; A61M 1/80; A61B 2017/22079; A61B 2017/22082

(57) ABSTRACT

A method of removing an occlusive material within a blood lumen of a patient includes positioning a material-removal device proximate to or in contact with the occlusive material within the blood lumen. The said material-removal device includes a catheter having a catheter lumen and a progressive cavity pump located at a distal portion of the catheter with the progressive cavity pump in fluid communication with the catheter lumen and the blood lumen. The method further includes actuating the progressive cavity pump to ingest at least a portion of the occlusive material from the blood lumen into the catheter lumen.

### CROSS-REFERENCE TO RELATED APPLICATIONS

This application is a continuation application under 35 U.S.C. §§ 120 and 365(c) of Patent Cooperation Treaty Application No. PCT/US2023/063580, filed Mar. 2, 2023, and titled “DEVICES AND METHODS FOR REMOVING MATERIAL FROM A PATIENT”.

PCT/US2023/063580 claims benefit of each of U.S. Provisional Pat. Application No. 63/315,764, which was filed Mar. 2, 2022, titled “DEVICES AND METHODS FOR ADMINISTERING AND REMOVING MATERIAL FROM A PATIENT”; U.S. Provisional Pat. Application No. 63/359,990, which was filed Jul. 11, 2022, titled “DEVICES AND METHODS FOR REMOVING UNWANTED MATERIAL FROM A PATIENT”; and U.S. Provisional Pat. Application No. 63/415,201, which was filed Oct. 11, 2022, titled “DEVICES AND METHODS FOR REMOVING UNWANTED MATERIAL FROM A PATIENT”.

The entire contents of each of the foregoing filings are incorporated herein by reference for all purposes.

### TECHNICAL FIELD

This disclosure relates generally to the field of medicine, and more specifically to the field of interventional radiology. Described herein are devices and methods for removing unwanted materials from a patient.

### BACKGROUND

The removal of materials from patients is an important part of routine and emergency medical care. For example, in the field of interventional radiology, removal of clot (which includes thrombus or thromboemboli) from blood vessels or artificial vascular grafts is known as thrombectomy and a variety of devices have been proposed to address this need. Limitations of some existing devices include but are not limited to significant amounts of blood loss during clot extraction, difficulty removing a variety of clot compositions including both soft and hard clot, difficulty removing clot adhered to the vessel wall, injury to blood vessels during clot extraction, large device size, clot fragmentation with subsequent embolization during removal, and the amount of capital equipment required to operate the devices. A device that can remove materials (e.g., clots, stones, malignant tissues) from body lumens or cavities which overcomes some or all of these limitations would be advantageous.

### SUMMARY

The present invention is directed to devices and methods for removing materials out of a patient. In one application, the devices and methods are used to remove a clot during a thrombectomy procedure. The device may include a tissue engagement portion, a pumping assembly, a catheter body, a handle assembly, and a collection assembly. When the device is activated, the clot is engaged by the tissue engagement portion, pulled into the pumping assembly, then progressively pumped into the catheter body, through the handle assembly, and into a collection assembly, thereby removing the clot from the blood vessel.

In one implementation, the pumping assembly is comprised of a rotor and a stator that form cavities or pockets that translate material through it and into the catheter body as the rotor rotates. In one implementation, the pumping assembly is a progressive cavity pump. In one example, the rotor extends into a tissue engagement portion at the distal end of the device. When activated, the tissue engagement portion creates a mechanical and/or vacuum-assisted engagement of the clot and material (e.g., blood and clot) such that they are ingested from the distal end of the tissue engagement portion and into the pumping assembly, which is configured to create sufficient pressure to move the material into the catheter body, through the handle assembly, and into a collection assembly. The tissue engagement portion may also aid in minimizing clot fragmentation and subsequent embolization. By engaging the clot, optimizing the size of the cavities, and controlling the rotational speed of the rotor, blood loss may also be minimized.

In some cases, the pumping assembly may further be configured to be positioned at a pre-defined or adjustable angle from the axis of the catheter body. In this case, the catheter body may be rotated via the handle assembly to better engage clot that is in larger vessels or adhered to the vessel wall. This may also allow for a smaller device to be utilized.

In some implementations, the pumping assembly may be slidably positioned within an engagement catheter to assist in ingesting material into the catheter.

The foregoing is a summary and may be limited in detail. The above-mentioned aspects, as well as other aspects, features, and advantages of the present technology are described below in connection with various implementations, with reference made to the description, claims and accompanying drawings.

### BRIEF DESCRIPTION OF THE DRAWINGS

FIG. 1A illustrates an implementation of a device according to the present disclosure in an isometric view.

FIG. 1B illustrates an exploded component view of the device of FIG. 1A.

FIG. 2A illustrates the device of FIG. 1A in a detailed view of the distal end.

FIG. 2B illustrates the device of FIG. 1A in an exploded component view.

FIG. 2C illustrates a front view of a pumping assembly with dimensions.

FIG. 2D illustrates a side view of a pumping assembly with dimensions.

FIG. 2E shows a representative flow rate equation of the pumping assembly.

FIG. 3A illustrates the device of FIG. 1A in an initial configuration with the stator and distal sleeve transparent.

FIG. 3B illustrates the device of FIG. 1A with the rotor partially rotated and a first cavity beginning to form.

FIG. 3C illustrates the device of FIG. 1A with the rotor further rotated.

FIG. 3D illustrates the device of FIG. 1A with the rotor further rotated and a second cavity beginning to form.

FIG. 3E illustrates the device of FIG. 1A with the rotor further rotated.

FIG. 3F illustrates the device of FIG. 1A with the rotor further rotated and a third cavity beginning to form.

FIG. 4 illustrates an implementation of the handle assembly of the device of FIG. 1A in an exploded component view.

FIG. 5 illustrates a section view of the handle assembly of FIG. 4.

FIG. 6A illustrates the device of FIG. 1A approaching a clot within a vessel.

FIG. 6B illustrates the device of FIG. 1A ingesting a clot.

FIG. 6C illustrates the device of FIG. 1A further ingesting a clot.

FIG. 6D illustrates the device of FIG. 1A further ingesting a clot.

FIG. 6E illustrates the device of FIG. 1A having ingested a clot.

FIG. 7 illustrates an implementation of a device according to this disclosure removing a clot from the iliac vein.

FIG. 8 illustrates an implementation of a method of use of the device of FIG. 1A.

FIG. 9A illustrates a pumping assembly that may be incorporated into implementations of the present disclosure.

FIG. 9B illustrates an alternate pumping assembly that may be incorporated into implementations of the present disclosure.

FIG. 10A illustrates another alternate pumping assembly that may be incorporated into implementations of the present disclosure.

FIG. 10B illustrates an exploded component view of the implementation shown in FIG. 10A.

FIG. 11A illustrates the device of FIG. 10A in a section view in an initial configuration.

FIG. 11B illustrates the device of FIG. 10A with the rotor partially rotated

FIG. 11C illustrates the device of FIG. 10A with the rotor further rotated

FIG. 12A illustrates an implementation of the device with aspiration and infusion channels.

FIG. 12B illustrates an exploded component view of the implementation shown in FIG. 12A.

FIG. 13A illustrates an implementation of the device with an even wall thickness stator.

FIG. 13B illustrates an exploded component view of the implementation shown in FIG. 13A.

FIG. 14A illustrates an implementation of a tissue engagement portion of a device according to this disclosure and including an extension feature.

FIG. 14B illustrates an implementation of an alternate tissue engagement portion including a stator funnel.

FIG. 14C illustrates an implementation of yet another tissue engagement portion including a rotor extension tip.

FIG. 14D illustrates an implementation of another tissue engagement portion including an extension wire.

FIG. 14E illustrates an implementation of another tissue engagement portion including a capped end.

FIG. 14F illustrates an implementation of another tissue engagement portion including a standoff.

FIG. 14G illustrates an implementation of another tissue engagement portion including an infusion outlet.

FIG. 14H illustrates an implementation of another tissue engagement portion including an infusion outlet.

FIG. 15A illustrates an implementation of a device according to this disclosure including a guidewire entry hole.

FIG. 15B illustrates a section view of the device of FIG. 15A.

FIG. 15C illustrates a side view of a device with multiple distal bends.

FIG. 16A illustrates a front view of a device according to this disclosure including a visualization assembly.

FIG. 16B illustrates an isometric view of the device in FIG. 16A.

FIG. 16C illustrates an exploded view of the device in FIG. 16B.

FIG. 17A illustrates a front view of a device according to this disclosure including two visualization assemblies.

FIG. 17B illustrates an isometric view of the device in FIG. 17A.

FIG. 17C illustrates an exploded view of the device in FIG. 17B.

FIG. 18A illustrates an aspiration catheter approaching a clot within a vessel.

FIG. 18B illustrates the aspiration catheter engaging the clot of FIG. 18A.

FIG. 18C illustrates a device according to this disclosure engaging with the clot of FIG. 18A.

FIG. 18D illustrates the device of FIG. 18C removing the clot of FIG. 18A.

FIG. 18E illustrates the clot of FIG. 18A removed.

FIG. 19A illustrates an implementation of a device according to this disclosure including a filter.

FIG. 19B illustrates the device of FIG. 19A with the filter shown in an exploded view.

FIG. 19C illustrates another implementation of a device according to this disclosure including a series of filters.

FIG. 20 illustrates an implementation of a device according to this disclosure with a catheter filter.

FIG. 21 illustrates an implementation of a device according to this disclosure with an expandable funnel.

FIG. 22 illustrates an implementation of a device according to this disclosure with a pumping assembly in a handle.

### DETAILED DESCRIPTION

Conventional and known removal devices include a variety of mechanisms and approaches to removing unwanted materials from a patient. Some devices include aspiration catheters which use a vacuum pump connected to a catheter to suck materials, such as thrombus, out of blood vessels like a straw. Aspiration catheters can be challenged by thicker materials such as subacute or chronic clot that do not easily compress into the catheter lumen and therefore can clog such devices. Additionally, aspiration catheters can deleteriously create significant aspirational flow rates and therefore remove large amounts of blood from the patient. Doing so increases the risk of exsanguination and the possibility of requiring a blood transfusion, among other risks.

Certain conventional removal devices have been described which use expandable metal frames and baskets to mechanically grab unwanted materials, like clot, in order pull the material from the blood vessel. These devices can sometimes cause vascular damage to the intimal wall, vein valves, or other tissue structures, particularly as they are removed from the patient. Additionally, these devices can sometimes allow fragments of the unwanted material to embolize and travel further downstream in the blood vessel, thereby increasing the risk of other issues, such as pulmonary emboli or strokes.

Still other conventional devices use spinning elements to macerate the unwanted material, which can include thrombus and wall adherent plaque. Devices with spinning elements can cause significant damage to vascular structures while also being prone to clogging, particularly as compared to other material removal devices and methods. These devices also often require large pieces of capital equipment for their control and may require a separate aspiration source in order to remove the material once the material has been macerated.

Described herein are devices and methods which overcome the previously described challenges, among others, while providing substantial additional benefits related to the removal of unwanted material from patients. During use, the disclosed device is generally inserted into a physiological lumen or cavity of a patient in proximity to material that is to be removed from the patient. In one aspect of the disclosed device, the device includes a pump for the generation of aspiration such that there is a low dead volume between the pump and the unwanted material to be removed. In some embodiments, the pump is a positive displacement pump which is situated within the lumen of the catheter and may even be located at the distal tip of the catheter such that the dead volume, or the fluid volume between the pump and the unwanted material in-situ, is minimized and possibly even negligible. By reducing the amount of dead space, the pump can provide a larger vacuum to the unwanted material due to minimal head losses through tubing. The pump can then discharge the unwanted material through the lumen of the catheter and out of the patient. Additionally, a benefit of positive displacement pumps is that they do not require large flow rates to generate high levels of vacuum and therefore can minimize blood loss associated with traditional aspiration catheters.

A positive displacement pump moves a fluid by repeatedly enclosing a fixed volume and moving it mechanically through the system in a cyclic motion. Examples of positive displacement pumps include piston pumps, diaphragm pumps, gear pumps, lobe pumps, vane pumps and progressive cavity pumps. While each of these pump types may be used for material removal and is within the scope of this disclosure, progressive cavity pumps (also known as Moineau pumps, eccentric screw pumps, or progressing cavity pumps) are especially well-suited for removing unwanted material from patients. A progressive cavity pump includes a stator with a slotted helical lumen and a spinning helical rotor. The stator and rotor are designed such that as they interface, one or more cavities are formed between the helical rotor and the stator helical lumen. As discussed below in further detail, the cavities can be contained volumes of fluid that may be fully sealed (e.g., due to an interference fit between the rotor and the stator, which may be facilitated by the stator being formed of an elastomeric material). Alternatively, this disclosure contemplates that the stator and the rotor may be sized such that the two components do not seal. Among other advantages, such “leaky” configurations can reduce the volume of certain fluids (e.g., blood) transferred by the progressive cavity pump while still facilitating uptake of solid/semi-solid materials and more viscous fluids. In the context of blood, the result can be reduced exsanguination and a lowered risk of the patient requiring a blood transfusion.

In certain implementations of this disclosure, the pitch of the helical rotor is about half the pitch of the stator helical lumen. Spinning the rotor causes the one or more cavities to move along the axis of the pump depending on which direction the rotor is spun. Progressive cavity pumps are unique from other pumps that incorporate helical elements such as Archimedes screw pumps, also known as water screw pumps. The typical design of an Archimedes screw pump includes a cylindrical shaft and about which a helical thread is wound and that spins within a cylindrical or tubular lumen. Such pumps do not include or form closed cavities between their stators and rotors and often utilize a pressure differential created by the rotational speed of the screw to aspirate fluid into the pump. Certain Archimedes screw designs may work well enough for pumping viscous fluid such as blood but may be challenged by thicker materials such as thrombus. By contrast, a progressive cavity pump forms a series of closed cavities that can either be fully or partially sealed. During operation, progressive cavity pumps move a discrete volume for each rotation of the rotor. As a result, the output of the progressive cavity pump is directly proportional to the number of rotations of the rotor and is independent of the rotational speed (as is the case of pressure-based pumps, such as conventional screw pumps). Progressive cavity pumps therefore have applications where the amount of pumped material needs to be well controlled, such as dosing applications. The controlled flow provided by progressive cavity pumps is also advantageous in the context of material removal as it overcomes some of the challenges associated with existing thrombectomy devices where excessive blood loss is difficult to control. Moreover, as briefly noted above, progressive cavity pumps are also well-suited for applications in which the pumped material is viscous or includes a slurry of solid materials, such as thrombectomy procedures, and can overcome some of the limitations with existing thrombectomy devices that clog with thicker materials. However, progressive cavity pumps tend to be too large, expensive, and complex than is feasible for use in a catheter. For example, conventional progressive cavity pumps are often used in large-scale applications, such as pumping of sewage water or oil. In such applications, the progressive cavity pumps have substantial mechanical complexity due to the need for various bearings and universal joints.

In certain implementations, the progressive cavity pump may further include elements that improve material removal. For example, the distal end of the pumping assembly may include a tissue engagement portion that is configured to ingest elastic and thick material into the pumping assembly by mechanically grabbing or pinching the material as well as applying aspiration force.

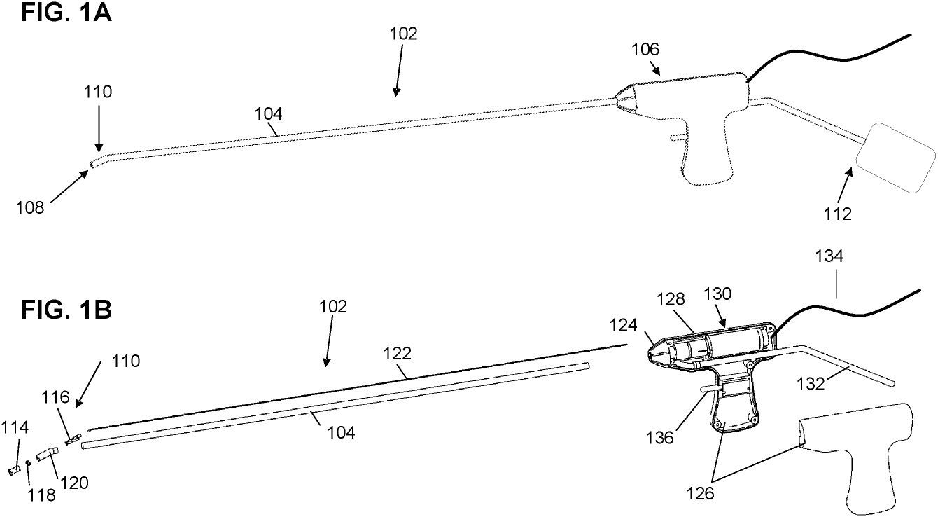

In FIG. 1A, a device 102 to remove material from a patient (e.g., from a blood vessel) is shown. The device 102 includes a tissue engagement portion 108, a pumping assembly 110, a catheter body 104, a handle assembly 106, and a collection assembly 112. The tissue engagement portion 108 is configured to engage the material via mechanical interaction with the material and/or via the generation of aspiration flow or vacuum pressure which draws the material into the tissue engagement portion 108.

FIG. 1B depicts additional details of the device 102. In the illustrated implementation, the pumping assembly 110 includes a stator 114, a rotor 116, a distal sleeve 120, and a locating element 118. The stator 114 fits within the distal sleeve 120, which is connected to the catheter body 104 and the locating element 118. The rotor 116 is attached to a torque member 122 and rotates together with it. The handle assembly 106 includes a catheter hub 124 for rotating the catheter body 104, a housing comprised of two shells 126, a drive system 130 to enable rotation of the torque member 122, a manifold 128 for sealing the drive system 130 from removed material and for directing the removed material through an outlet tube 132 to a collection assembly 112. The handle assembly 106 further includes an electrical cable 134 and controller 136.

In one implementation, the pumping assembly 110 is a progressive cavity pump, where a series of cavities is formed between the rotor 116 and stator 114 that move proximally as the rotor 116 spins one direction and move distally as the rotor 116 spins the opposite direction. Progressive cavity pumps are known by other names such as eccentric screw pumps, progressing cavity pumps, or Moineau pumps. Progressive cavity pumps are well suited in applications requiring fluid metering, pumping viscous or shear-sensitive materials, and pumping slurries containing abrasive solids.

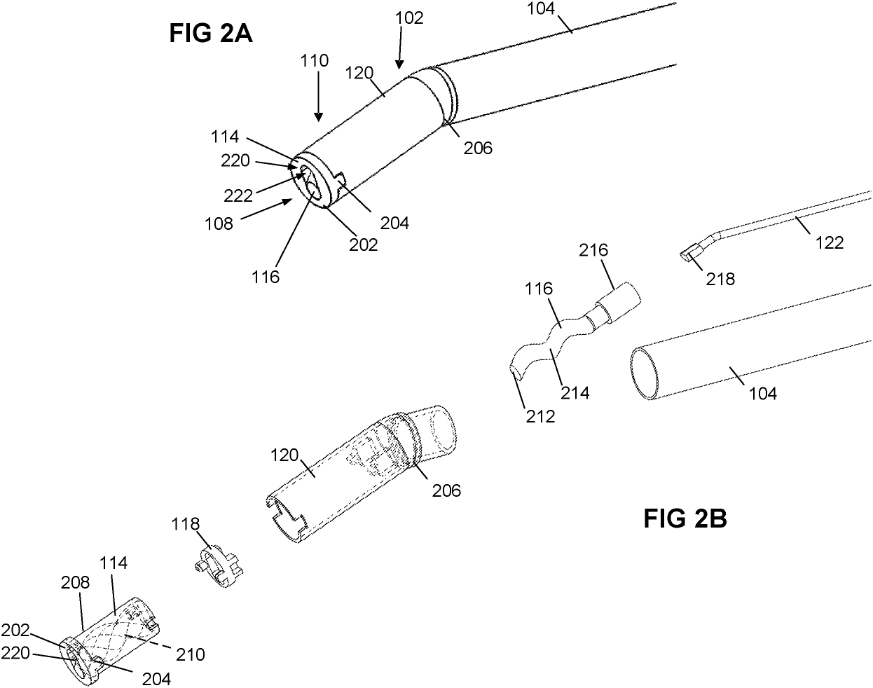

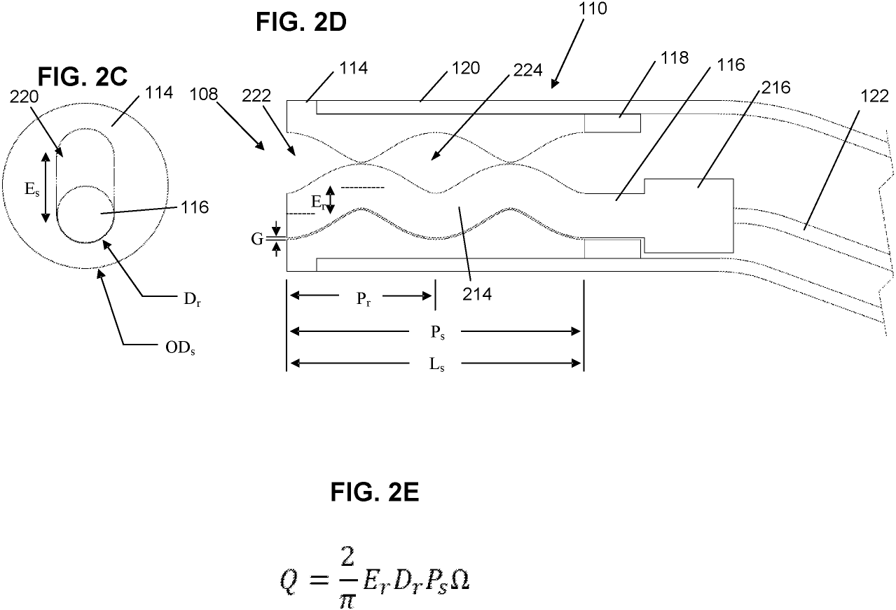

In FIG. 2A, a detailed view of the distal end of device 102 is shown and further illustrates the tissue engagement portion 108 and the pumping assembly 110. Generally speaking, the tissue engagement portion 108 includes elements that mechanically engage tissue to facilitate subsequent ingestion of the tissue by device 102. In certain implementations, the tissue engagement portion 108 includes a rotor distal tip 212 (indicated in FIG. 2B) of the rotor 116, a stator distal section 202 of the stator 114, a slot 220 defined by the stator 114, and portions of the distal sleeve 120.

In the illustrated implementation, the stator distal section 202 extends from the distal sleeve 120 and is oriented with a stator alignment feature 204. The stator alignment feature 204 provides rotational orientation between the stator 114 and the distal sleeve 120. The slot 220 allows for the rotor distal tip 212 of the rotor 116 to move within it and creates an open cavity 222 at the distal end of the device 102. As the rotor 116 rotates, the rotor distal tip 212 spins and translates across the slot 220. This action mechanically engages material (e.g., by pinching or entrapping the material between the rotor distal tip 212 and the walls defining the slot 220). The pumping assembly 110 may also generate aspiration flow and/or vacuum pressure to pull tissue/material into the open cavity 222. By doing so, the device 102 ingests material into the pumping assembly 110 for subsequent removal from the patient.

In certain implementations, the distal sleeve 120 may include a distal bend 206 that orients the distal sleeve 120 at an angle relative to the catheter body 104. The distal bend 206 may enhance the ability of the tissue engagement portion 108 to engage material (e.g., a clot) that is adhered to a vessel wall and/or increase the reach of the device 102, e.g., to treat larger vessels without needing a larger diameter device. In certain implementations, the distal bend 206 may be adjustable or shapeable. For example, the catheter body 104 may include one or more steering features (e.g., pull cables) that may be used to steer and lock the distal bend 206.

As previously noted, the stator 114 includes the slot 220 (which may be obround in shape) at its distal face. The slot 220 continues helically through the stator 114 to form a stator helical lumen 210. In certain implementations, the stator 114 can be formed from an elastic material, e.g., an elastomer, such as silicone, polyurethane, or any other suitable material which can be molded or otherwise formed to include stator helical lumen 210. Among other things, using an elastomer facilitates sealing of the stator 114 against the rotor 116. In certain non-limiting examples, the stator 114 material can have a durometer from and including about Shore 20A to and including about Shore 100D, or from and including about Shore 20A to and including about Shore 60D, or about Shore 80A. In other implementations, the stator can be comprised of a more rigid material such as plastic, stainless steel, or any other suitable material.

The rotor 116 generally has a rotor helical section 214 that has a geometry similar to that of the stator helical lumen 210 albeit with a reduced pitch. For example, in certain implementations, the pitch of the rotor helical section 214 may have approximately half the helical pitch of the stator helical lumen 210 (or, stated differently, the stator helical lumen 210 may have about double the pitch of the rotor helical section 214). Alternate geometries of the stator helical lumen 210 and the rotor helical section 214 can be configured with similar functionality and will be discussed further later in this disclosure.

The rotor 116 can be formed from various materials including, but not limited to, nylon, polycarbonate, stainless steel, nitinol, or any other suitable material (e.g., other plastics or metals). In certain implementations, the rotor 116 may be formed from plastic materials and can be formed using one or more of injection molding, 3D printing, extrusion, or similar manufacturing methods. In general, however, the rotor 116 may be formed from a material stiffer or more rigid than that of the stator 114. Among other things, selecting a stiffer/mode rigid material for rotor 116 and a more elastic/compliant material for the stator 114 facilitates sealing between the rotor 116 and the stator 114 during operation.

The geometries of the stator 114 and the rotor 116 generally form a path of contact or near contact that is helical. During operation, at least one closed cavity 224 is formed in the space between the rotor helical section 214 and the stator helical lumen 210. More specifically, when the rotor 116 is rotated within the stator 114, cavities are formed between the rotor 116 and the stator 114 and travel proximally toward catheter body 104 as the rotor 116 is rotated. For example, FIG. 2D illustrates each of an open cavity 222 and a closed cavity 224. During operation, material is initially captured within the open cavity 222. As the rotor 116 is further rotated, the open cavity 222 moves proximally and is eventually closed, forming a closed cavity, such as the closed cavity 224. As the rotor 116 is further rotated relative to the stator 114, the closed cavity 224 eventually progresses to the proximal end of stator helical lumen 210 and opens, releasing the captured material into catheter body 104. In this way, material drawn into pumping assembly 110 is captured within the closed cavity 224 and subsequently moved into the catheter body 104 for removal from the patient. Among other advantages, this approach, allows for removal of material (e.g., clots) without removing excessive amounts of fluid (e.g., blood).

In one implementation, the closed cavity 224 is fluidically sealed. For example, sealing can be achieved by contact between the stator 114 and the rotor 116 and, in some implementations in which the stator 114 and the rotor 116 have an interference fit, by additional compression of the stator 114 by the rotor 116. By way of example, compression of the stator 114 by the rotor 116 can be from and including about 0% to and including about 40%, or from and including about 2% to and including about 10%, or about 5%. The compression need not be the same along the entire length of the contact between the stator 114 and the rotor 116.

In other implementations, closed cavities formed between the stator 114 and the rotor 116 may not be fluidically sealed, i.e., a clearance gap may be present between the rotor 116 and stator 114. Even without fully sealed cavities, the pumping assembly 110 may still perform similarly as with sealed cavities, particularly for removing solid material. In certain implementations, the clearance gap may beneficially lower friction between the stator 114 and the rotor 116 due to the lack of contact or elastic interference between the two components. A clearance gap may also allow for larger tolerance variations of the rotor 116 and stator 114 and may enable both components to be made of rigid material. Including a clearance gap may also reduce the flow rate through pumping assembly 110, e.g., by permitting distal leakage of fluid from the pumping assembly 110. Among other advantages, such leakage can result in removal of solid or semi-solid material from a patient without removing substantial amounts of blood or other fluids. By way of example, the ratio of removed blood to removed clot can be from and including about 1:10 to and including about 50:1, or from and including about 1:1 to and including about 20:1, or about 10:1. For purposes of this disclosure and unless otherwise specified, cavities described as being “closed” within this disclosure should be considered to encompass both fluidically sealed and unsealed cavities.

The pumping assembly 110 shown includes a distal sleeve 120 for containing the stator 114 and may provide support such that the stator 114 may be compressed between the rotor 116 and distal sleeve 120 to permit formation of closed cavities, such as the closed cavity 224. The stator 114 generally includes a stator body section 208 that is sized to fit within the distal sleeve 120. The distal sleeve 120 may be an integral part of the catheter body 104 or may be a separate part which is attached to the catheter body 104 as shown in FIG. 2B. The distal sleeve 120 connects to the catheter body 104 such that the stator distal section 202 maintains a tubular profile.

Certain implementations include a distal bend, the distal bend 206 in the distal sleeve 120 generally angles the tip of the device away from the axis of the catheter body 104. The angle of the distal bend 206 can be from and including about 0° to and including about 75°, or from and including about 10° to and including about 45°, or about 20°.

In certain implementations, the distal sleeve 120 may be a laser cut stainless steel or nitinol tube, a plastic tube, an injection molded component, a machined component, or have any other suitable construction.

In certain implementations, the pumping assembly 110 may include a locating element 118 that resides inside the distal sleeve 120 and engages with the stator 114 and a rotor hub 216 such that the axial position of the rotor 116 is maintained relative to the stator 114. The locating element may include a distal hard stop, a proximal hard stop, or other alignment features that position the rotor 116 or stator 114 both axially and rotationally. Among other things, the locating element 118 ensures that the distal end of the rotor 116 is in the correct axial location and in some implementations does not extend beyond the distal end of the stator distal section 202, which may lead to vessel injury. The locating element 118 can further engage with internal features (e.g., shoulders, slots, protrusions, keys, keyways, etc.) within the distal sleeve 120 such that the position and orientation of the stator 114 relative to the distal sleeve 120 is maintained. In some implementations, the locating element 118 is a separate component and in other implementations the locating element 118 is a feature (e.g., integrally formed or coupled to) of the stator 114 or distal sleeve 120.

The torque member 122 is connected to the rotor 116 (e.g., via the rotor hub 216) such that when the torque member 122 spins, the rotor 116 spins as well. The torque member 122 can be any number of constructions such as wire rope, cables, tubes, wire, or any other suitable construction to spin the rotor 116. More generally, torque member 122 has a structure and/or is formed of a material such that torque member 122 has sufficient torsional rigidity to transfer torque to the rotor 116 while retaining sufficient bending flexibility to facilitate navigation through physiological lumens of a patient. The torque member 122 may be constructed differently depending on the specific application to accommodate the need for different flexibilities. For example, in applications involving more tortuous or stiffer physiological lumens, a more flexible torque member may be used while a generally stiffer torque member may be used in applications in which the physiological lumen of the patient is straighter, larger, or more flexible. The torque member 122 can include a torque member crimp 218 at one end which facilitates a connection to the rotor 116 (e.g., to the rotor hub 216). In some cases, the rotor 116 and torque member 122 are a single component with the rotor 116 shaped into, integrally formed with, or coupled to the torque member 122.

The device 102 may further include any number of radiopaque materials such as marker bands or components comprised of radiopaque materials such that the device 102 is visible using fluoroscopy or computed tomography (CT) imaging. For example, the distal end of the device 102 may include a band or strip of material such as tantalum, platinum, iridium, titanium, gold, tungsten, stainless steel, or any other suitable radiopaque material. The marker band may be placed near the distal tip of the device 102 to provide feedback to the user on the location of the tip. In other implementations, the stator 114 or rotor 116 or distal sleeve 120 may be comprised of a material with radiopaque additives such as barium sulfate, bismuth, tungsten, or any other suitable additive.

FIGS. 2C and 2D illustrate additional views of pumping assembly 110 and further indicate specific dimensions. In general, the size of the device 102 may be adapted for any number of applications and be configured (e.g., sized) for specific applications. For example, the device 102 may be configured for thrombectomy of deep vein thrombosis. Due to the relatively large vessels and possible amount of clot involved in such procedures, implementations may include configurations in which the outer diameter of the stator ODₛ and the catheter body 104 may be from and including about 1 mm (3 Fr) to and including about 8 mm (24 Fr). In one specific example, the outer diameter ODₛ can be about 5.3 mm or 16 Fr. The length of the stator Lₛ may also vary in different applications. However, by way of non-limiting example, in applications directed to treatment of deep vein thrombosis, Lₛ be from and including about 1 mm to and including about 30 mm or about 10 mm.

The total number of rotations of the stator lumen may also vary. For example, in certain implementations, the stator helical lumen 210 may include from and including about 0.5 revolutions to and including about 10 revolutions or about 1 revolution of the stator helical lumen 210.

The cross-sectional diameter of the rotor Dᵣ can also vary. However, in certain implementations Dᵣ may be from and including about 0.1 mm to and including about 4 mm or about 2 mm.

In the implementation shown in FIGS. 2A-2D, the rotor helical section 214 has a circular profile, however, other geometries are contemplated and within the scope of this disclosure.

The stator helical lumen 210 can have a stator pitch Pₛ and the rotor helical section 214 can have a rotor pitch P, that is fractionally shorter than Pₛ. For example, Pᵣ may be from and including about 0.25 times to and including about 0.75 times or about 0.5 times Pₛ. For example, the rotor pitch Pᵣ may be about 5 mm while the corresponding stator pitch Pₛ may be about 10 mm while the length Lₛ of both helical elements may be about 10 mm. In such a configuration, the stator helical lumen 210 would have about 1 full revolution while the rotor helical section 214 would have about 2 revolutions.

The stator slot 220 may be defined by an eccentricity Eₛ. In certain non-limiting examples, the eccentricity Eₛ may be from and including about 0.1 mm to and including about 4 mm or about 2 mm. Similarly, the rotor helical section 214 may be defined by an eccentricity Eᵣ. The rotor eccentricity Eᵣ may be a fraction of the stator eccentricity Eₛ. In certain implementations, the rotor eccentricity Eᵣ may be from and including about 0.25 times to and including about 0.75 times or about 0.5 times the eccentricity of the stator slot Eₛ. In the illustrated implementation, for example, the rotor eccentricity Eᵣ can be about 1 mm while the stator slot eccentricity Eₛ can be about 2 mm.

As previously noted, and as shown in FIG. 2D, the stator 114 and the rotor 116 may be selected to have a clearance gap G between them. In some implementations, the rotor 116 and stator 114 have an intentional interference to provide a fully sealed closed cavity 224. In such cases, the clearance gap G may be characterized by a negative value. For example, the clearance gap G may be from and including about -0.01 mm to and including about -0.5 mm or about -0.05 mm. The negative clearance gap G can alternatively be expressed as a percent of compression as described above.

In implementations where there is an intentional positive clearance gap G between the rotor 116 and the stator 114, the clearance gap G may be characterized by a positive value. For example, the clearance gap G may be from and including about 0.0 mm to and including about 1.0 mm or from and including about 0.05 mm to and including about 0.25 mm or about 0.01 mm. In such implementations, the cavity created between the stator 114 and rotor 116 is an unsealed cavity. Among other things, a positive clearance gap G (e.g., a non-interfering fit between the stator 114 and the rotor 116) can result in lower friction between the stator 114 and the rotor 116, making rotation of the rotor 116 easier and may allow both the rotor 116 and stator 114 to be comprised of more rigid materials.

In the implementation shown, each closed cavity 224 formed between the stator helical lumen 210 and the rotor helical section 214 has a respective volume. Although the volume of each cavity can vary depending on the size and geometry of the components of device 102, in at least certain implementations, the volume of each cavity may be from and including about 1 µl to and including about 200 µl, or about 17 µl.

Since each cavity is formed during a rotation of the rotor 116 and represents a fixed volume defined by the space between the rotor helical section 214 and the stator helical lumen 210, the flow rate is approximately proportional to the rotational speed of the torque member 122 which can be adjusted by the user accordingly. FIG. 2E provides an equation for the approximate flow rate Q of the pumping assembly 110 for a given rotational speed of the rotor Ω in radians per second. For example, in the implementation shown, the rotor eccentricity Eᵣ is 1 mm, the rotor diameter Dᵣ is 2 mm, and the stator pitch Pₛ is 10 mm. Based on a rotational speed Ω of 1,000 rpm or 104 rad/s, the flow rate Q is estimated to be approximately 40 cc/min. The design parameters can be adjusted as necessary to alter the properties of the pump assembly including the flow rate Q, pump pressure, and other characteristics. The rotational speed of the drive system 130 may range from and including about 1 rpm to and including about 120,000 rpm, or from and including about 2,000 rpm to and including about 60,000 rpm, or about 15,000 rpm.

A positive clearance gap between the stator 114 and the rotor 116 may reduce the flow rate of the pumping assembly 110 by allowing fluid to slip between the clearance gaps. This may beneficially reduce evacuated viscous materials, such as blood, while still ingesting thicker or solid/semi-solid materials. In this manner, the device 102 may be more preferential in removing thicker materials. The equation in FIG. 2E does not account for fluid slippage that may exist if the closed cavity 224 is an unsealed cavity due to a positive clearance gap between the rotor 116 and stator 114; however, such adjustments can be made, e.g., computationally or based on empirical data. Notably, implementations including positive clearance gaps may not generate significant vacuum pressure, further affecting flow and ingestion characteristics of the pumping assembly 110.

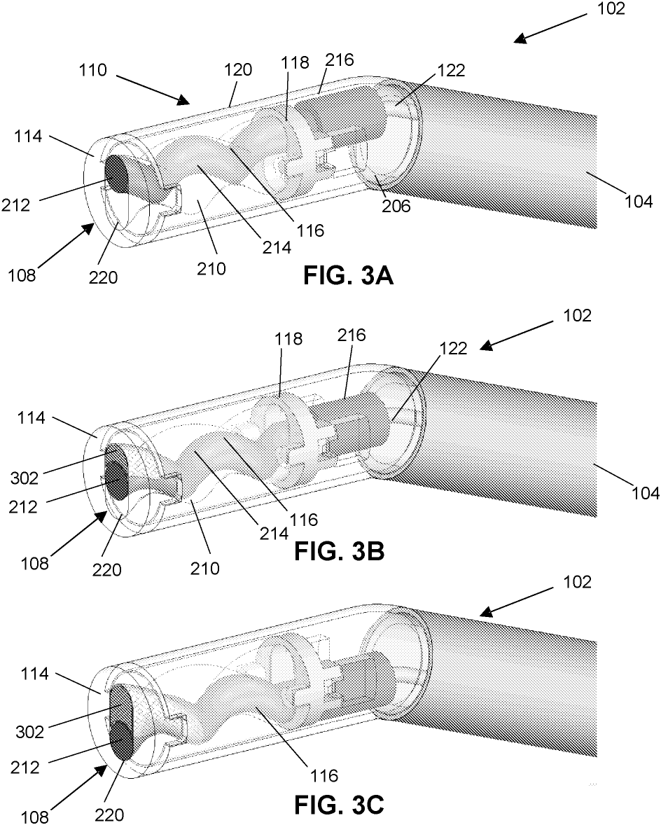

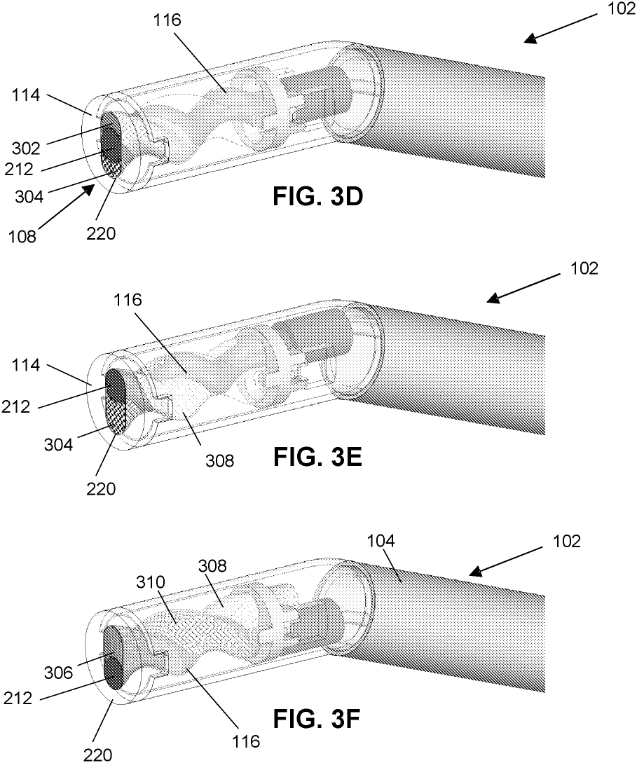

In FIGS. 3A-3F, the operation of the pumping assembly 110 is shown in more detail by making the distal sleeve 120 and stator 114 transparent to observe the motion of the cavities. In FIG. 3A, the device 102 is shown in an initial configuration. The stator 114 is within the distal sleeve 120 and the rotor helical section 214 is within and the stator helical lumen 210. The rotor hub 216 is pressed against the locating element 118 which is at the proximal end of the stator 114. The torque member 122 is connected to the rotor 116 and moves through the distal bend 206 into the catheter body 104. In this initial configuration, the rotor distal tip 212 is at the top of the slot 220. In certain applications, the pumping assembly 110 may be already primed with fluid such as water, saline, blood, grease, or oil, or conversely the pumping assembly 110 may be empty of material.

FIG. 3B is an illustration of the device 102 in a partially rotated position with the torque member 122 and the rotor 116 partially rotated. As previously discussed, the rotor helical section 214 interacts with the stator helical lumen 210 as the rotor 116 turns such that the rotor helical section 214 is in or near contact with the stator helical lumen 210 to define cavities via which material is transported through pumping assembly 110.

As shown, the rotor hub 216 interacts with the locating element 118 to ensure that the rotor 116 is in the correct axial location. More specifically, as the rotor 116 is rotated clockwise, interaction between the rotor 116 and stator 114 results in a distally directed force on the rotor 116. The locating element 118 provides that limits distal travel of the rotor hub 216 and keeps the rotor distal tip 212 in the desired location at the tissue engagement portion 108. In one implementation, the rotor distal tip 212 does not extend significantly beyond the stator distal section 202 to reduce risk of vessel injury. As the rotor 116 rotates, the rotor distal tip 212 moves down within the slot 220 at the distal end and a first open cavity 302 is formed in the space between the rotor 116 and the slot 220. The first open cavity 302 is open to the distal end of the pumping assembly 110 allowing fluid or material to enter into the stator 114. The material may be blood or clot or any other material to be removed.

As shown in FIG. 3C, as the rotor 116 is further rotated, the volume of the first open cavity 302 increases as the first open cavity 302 moves proximally into the pumping assembly 110. In certain implementations, this action creates a negative space that draws in fluid like a syringe. In the case of clot material, the tissue engagement portion 108 can additionally create mechanical forces on the tissue, such as by grabbing or pinching the tissue, and pull it into the first open cavity 302. Notably, in FIG. 3C, the torque member 122 and rotor 116 have rotated further such that the rotor distal tip 212 is at the bottom of the slot 220. The first open cavity 302 has continued to increase in size and move proximally along the length of stator 114, further drawing in fluid or material with either aspirational or mechanical forces.

FIG. 3D illustrates the device 102 following further rotation of the rotor 116 (as compared to FIG. 3C), which moves the rotor distal tip 212 upward and towards its initial position in the slot 220. As shown., the opening to the first open cavity 302 has decreased as the first open cavity 302 moves into the pumping assembly 110. At the same time, a second open cavity 304 has opened at the distal end of the device 102 and fluid or material begins being drawn into second open cavity 304. In the illustrated implementation, the first open cavity 302 and the second open cavity 304 are separate cavities that are formed by the contact created between the rotor 116 and the stator 114 and there is limited or no fluid connection between the first open cavity 302 and the second open cavity 304. In other implementations where a clearance gap exists between the rotor 116 and stator 114, there may be small fluid connection between the first open cavity 302 and second open cavity 304.

FIG. 3E illustrates the device 102 following further rotation of the rotor 116 (as compared to FIG. 3D) such that the rotor distal tip 212 is back to the initial configuration in the slot 220 shown in FIG. 3A. As illustrated, the first open cavity 302 shown in the previous figures has transitioned into a first closed cavity 308 that is no longer exposed to the distal opening of the slot 220 and which moves proximally along the pumping assembly 110 within the stator 114 as the rotor 116 continues to be rotated. Stated differently, the first closed cavity 308 is a formed volume within the stator 114 that translated along the axis of the pumping assembly 110. In the illustrated implementation, the first closed cavity 308 is not fluidly connected to either the external environment at the distal end of the pumping assembly 110 or the lumen of the catheter body 104. As further illustrated in FIG. 3E, the second open cavity 304 has become more open to the distal end of the pumping assembly 110 and, as a result, may draw in fluid or material using vacuum and/or mechanical forces.

FIG. 3F illustrates the device 102 following further rotation of the rotor 116 (as compared to FIG. 3E). More specifically, the rotor 116 has been rotated another half-turn such that the rotor distal tip 212 returns to the bottom of the slot 220. As illustrated, the first closed cavity 308 has further moved proximally to be in communication with the internal volume of the catheter body 104. Stated differently, the first closed cavity 308 is now urged into the distal sleeve 120 and upon further rotation of the rotor 116 the fluid or material within the first closed cavity 308 will be expelled out of the pumping assembly 110 and into the catheter body 104. As further illustrated in FIG. 3F, the second closed cavity 310 has become fully contained within the stator 114 (e.g., is no longer open at the distal end of the pumping assembly 110). Finally, a third open cavity 306 has been formed at the distal end of the device 102 between the rotor helical section 214 and the stator helical lumen 210, permitting ingestion of material into the third open cavity 306.

In the manner illustrated in FIGS. 3A-3F and discussed above, the rotor 116 can continue to rotate such that additional cavities form at the distal end of the device 102, translate through the pumping assembly 110, and open into the catheter body 104, each cavity carrying with them fluid and/or solid or semi-solid material. Stated differently, fluid and material can be pumped from the external environment through the pumping assembly 110 and into the catheter body 104 by virtue of the pumping action of the pumping assembly 110.

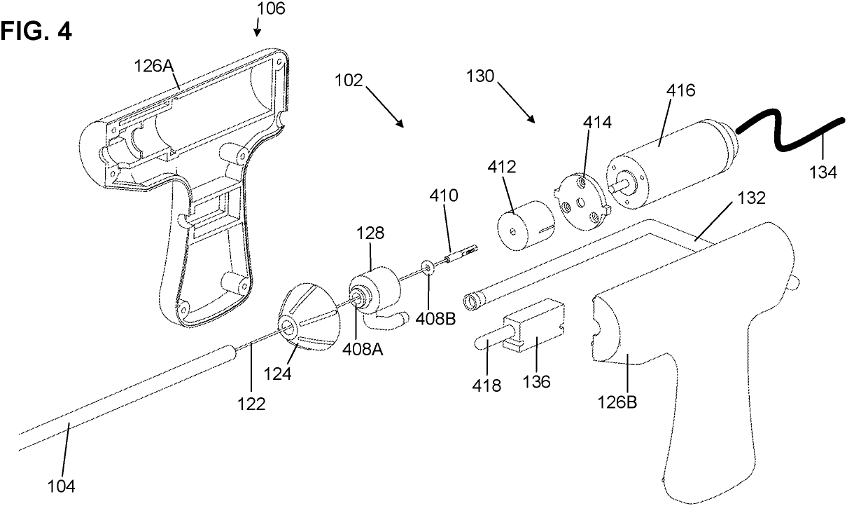

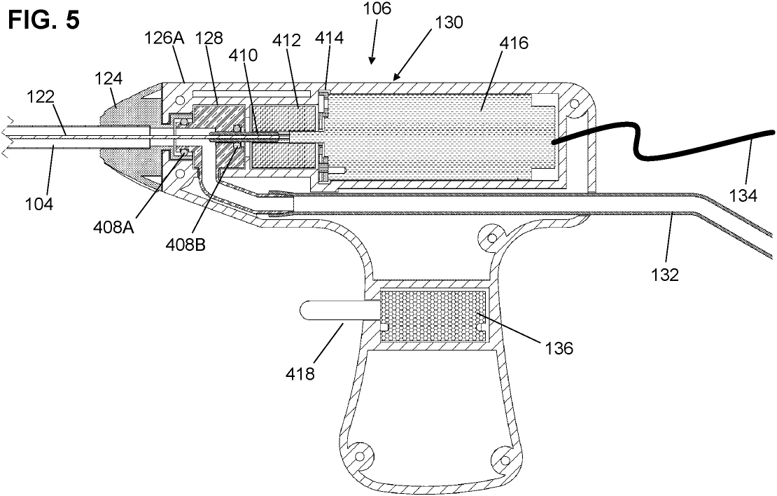

Turning now to FIGS. 4 and 5, an example of an implementation of a handle assembly 106 for use with devices according to this disclosure is shown. Among other things., the handle assembly 106 allows a user to control certain aspects of the device 102 function which will be described in more detail, below. In the implementation shown, the handle assembly 106 includes a catheter hub 124, a drive system 130, a manifold 128, and a housing including two handle shells 126A, 126B. The catheter hub 124 is connected to the catheter body 104 and can be rotated by the user to rotate the catheter body 104 and in turn, the distal sleeve 120, locating element 118, and stator 114. Such rotation may be particularly useful in directing the tissue engagement portion 108 with and distal bend 206 toward adhered tissue (e.g., an adhered clot) or particular areas of a vessel. This feature effectively increases the reach of the device 102 allowing for larger diameter vessels to be treated without the need for a larger device.

The drive system 130 includes a torque member 122, a torque member hub 410, a motor adaptor 412, a motor plate 414, a motor 416, a throttle 418, a controller 136, and an electrical cable 134. The torque member 122 is housed within the catheter body 104 and enters the handle assembly 106 to engage the drive system 130. The torque member 122 passes through the catheter hub 124 and into a manifold 128. The manifold includes two sealing members 408A, 408B, which prevent fluid and material from entering the drive system 130. The proximal end of the torque member 122 includes a torque member hub 410 which is configured to be rotated by the motor 416. The motor 416 is attached to a motor plate 414 which holds it within the handle shells 126A, 126B. A motor adaptor 412 connects the shaft of the motor 416 to the torque member hub 410 such that rotation of the motor 416 causes rotation of the torque member 122. An electrical cable 134 provides electrical power to the motor 416 and may possibly include control inputs or outputs to the device 102. In other implementations, the electrical cable 134 may be replaced with a battery inside the handle shells 126A, 126B that powers the drive system 130 instead of an external power source. The handle assembly 106 further includes a throttle 418 and controller 136 that allows the user the control aspects of the actuation and/or speed of the motor 416.

An outlet tube 132 is connected to the manifold 128 and directs fluid and material out of the handle assembly 106 and into a collection assembly 112 (shown in FIG. 1A) located outside the handle assembly 106. In other implementations, the device 102 can collect the fluid and material in a collection assembly 112 located within handle assembly 106 or catheter body 104. In still other implementations, the outlet tube 132 or collection assembly 112 may incorporate a filter to separate clot material from blood and thereby allow the reintroduction of the blood into the patient.

In certain implementations, the drive system 130 may be manually operated. For example, the motor adaptor 412, motor plate 414, motor 416, electrical cable 134, and throttle 418 may be replaced with mechanisms (e.g., knobs, cranks, handles, gears, etc.) to facilitate manually driving rotation of the torque member 122.

In FIG. 5, is a cross-sectional view of the handle assembly 106. The catheter hub 124 extends from the front of the housing shells 126A, 126B (shell 126B is shown in FIG. 4) and allows the user to turn the catheter body 104 to direct the tissue engagement portion 108 (shown, e.g., in FIG. 1A) around the circumference of a vessel, as previously described. The sealing members 408A, 408B, which are housed within or disposed on opposite ends of the manifold 128, prevent leaking of fluid from the manifold 128. For example, the front sealing member 408A may be engaged with the catheter hub 124 so that as the catheter hub 124 is rotated, the front sealing member 408A maintains a fluid seal within the manifold 128. The rear sealing member 408B may be engaged with the torque member hub 410 to prevent leaking as the torque member hub 410 spins. As shown in FIG. 5, the torque member hub 410 and motor 416 are connected by the motor adaptor 412. The outlet tube 132 connects to the manifold 128 and exits from the handle shells 126A, 126B to a collection assembly 112 (shown in FIG. 1A). The throttle 418 and controller 136 may be contained within and held by the handle shells 126A, 126B, e.g., in a handle or grip portion of the handle assembly 106.

In the implementation shown, the throttle 418 is a spring return potentiometer that changes resistance as the user depresses the extending plunger. Wires from the potentiometer (not shown) connect to the electrical cable 134. The electrical cable 134 can go to a power supply or mains power. In some implementations the controller 136 may exist outside the handle assembly 106 and is adapted to control the motor 416 rotation and speed through the electrical cable 134.

The handle assembly 106 shown in FIGS. 4 and 5 are intended merely as a non-limiting example of a handle that may be used in implementations of this disclosure. In general, handle assemblies according to this disclosure allow for actuation of the pumping assembly, e.g., by causing rotation of the rotor within the stator of the pumping assembly. As noted above, further functionality provided by the handle may include, but is not limited to, rotating the catheter body and providing a channel through which captured fluids and material may be transported, filtered, or otherwise processed. Accordingly, while FIGS. 4 and 5 provide an example implementation of a handle assembly, handle assemblies and related control hubs for devices according to this disclosure are not limited to the specific configuration shown in FIGS. 4 and 5.

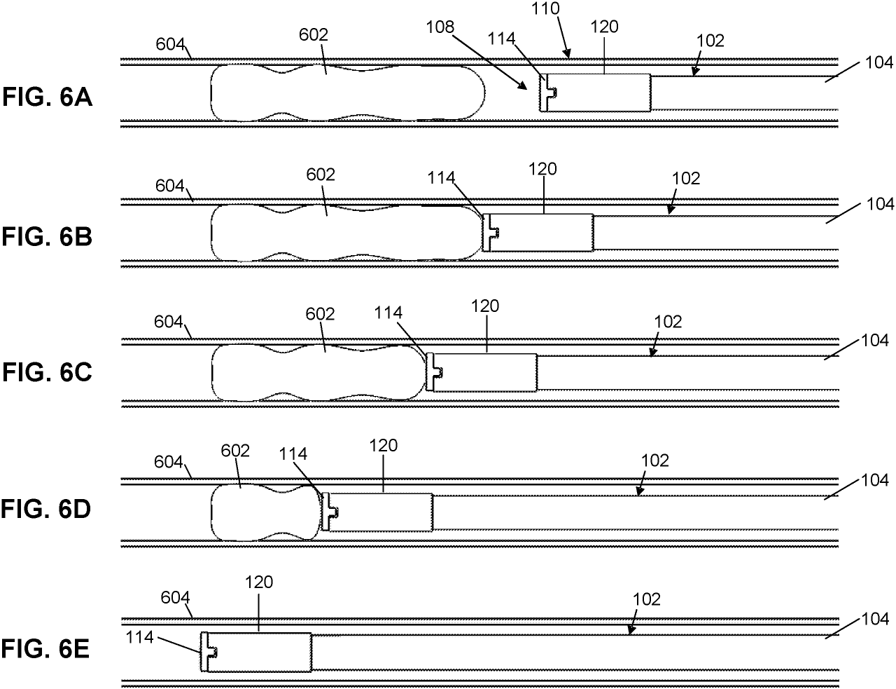

FIGS. 6A-6E illustrate the device 102 at various stages in use for a clot removal application and, in particular, a thrombectomy procedure. For example, the process shown in FIGS. 6A-6E may correspond to the removal of thrombus for a patient suffering from deep vein thrombosis where a clot is in the iliofemoral vein or a pulmonary embolism where a clot is in the pulmonary artery and blocking normal blood flow. The procedure illustrated in FIGS. 6A-6E is provided merely as an example and this disclosure recognizes that the general process and functions illustrated in FIGS. 6A-6E may be readily adapted to other clinical applications.

In FIG. 6A, the device 102 is shown within a blood vessel 604 that has a clot 602 which is partially or completely blocking blood flow. In certain applications and processes, the device 102 can be inserted into the patient’s vasculature and navigated to the blood vessel using standard catheterization techniques and devices.

At the point illustrated in FIG. 6A, the user can actuate the device 102, e.g., by depressing the throttle 418 of the handle assembly 106 (shown in FIGS. 4-5). Actuating the device 102 causes rotation of the torque member 122, which in turn causes the rotor 116 to rotate, thereby activating the tissue engagement portion 108 and the pumping assembly 110, e.g., as described in FIGS. 3A-3F. The tissue engagement portion 108 can engage the clot 602 and begin ingesting blood and material through the pumping assembly 110 and into the catheter body 104 and outlet tube 132 (shown, e.g., in FIG. 1A).

In some implementations, the pumping assembly 110 can create vacuum pressures or aspiration flow within the vessel 604 that can urge the clot 602 proximally. In some cases, the vacuum pressure can be from and including about 0.5 inHg to and including about 29.2 inHg. Alternatively, the device 102 can be advanced forward (e.g., toward the clot 602) until the tissue engagement portion 108 is in contact with the clot 602, as shown in FIG. 6B.

In FIG. 6B, the device 102 is advanced such that the tissue engagement portion 108 is in contact with the clot 602. As the rotor 116 continues to rotate, pieces of the clot 602 can be pulled into the slot 220 by aspiration flow or mechanical forces (e.g., grabbing or pinching the clot 602 or pieces of the clot 602) and subsequently into the open cavities that are formed by the tissue engagement portion 108 and pumping assembly 110, as previously discussed. The tissue engagement portion 108, consisting of the tissue contacting elements such as the slot 220 and rotor distal tip 212, can both mechanically grab the clot and/or mobilize it through aspiration. As the cavities move proximally, the clot 602 is likewise moved through the pumping assembly 110 and expelled through the catheter body 104 and outlet tube 132 and into the collection assembly 112.

In FIG. 6C, the device 102 has removed a portion of the clot 602. The removed portion is within the catheter body 104 and being pumped out of the patient, as previously described. In FIG. 6D, the device 102 is further advanced and has removed more of the clot 602 from the blood vessel 604 by the tissue engagement portion 108 and has ingested the clot 602 into the device 102. In FIG. 6E, the clot 602 has been fully removed from the blood vessel 604 and has been removed from the patient, e.g., by transporting the clot 602 through the catheter body 104, through the handle assembly 106, and into the collection assembly 112 coupled to the handle assembly 106.

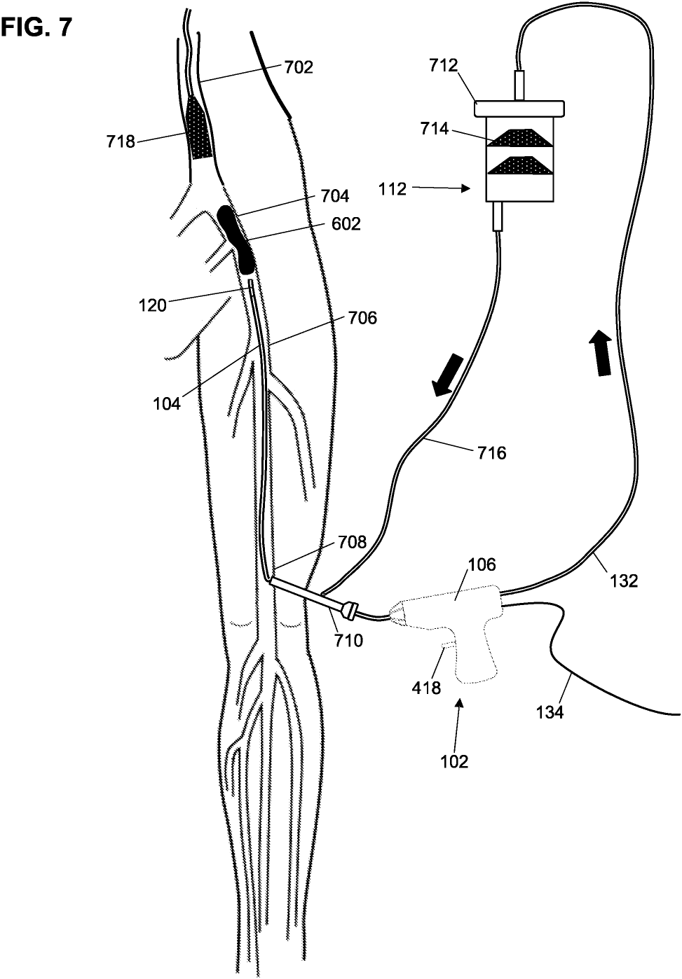

In FIG. 7, an implementation of the device 102 is shown in an exemplary clinical application. A clot 602 has formed within the iliac vein 704 of a patient. The clot 602 restricts blood flow within the vessel creating leg swelling and pain for the patient in the patient’s lower extremities. Prior to inserting the device 102, an embolic distal protection element 718 can optionally be placed within the inferior vena cava 702 for protection against any dislodged or fragmented portions of the clot 602 from traveling to the heart and lungs. In one specific implementation, the distal protection element 718 can be an expandable container such as a braid that catches clot fragments but allows for blood flow. Alternatively, the distal protection element 718 may include a non-porous element such as a balloon or membrane which completely arrests blood flow. The distal protection element 718 can be inserted through the jugular vein or other appropriate access site and directed into the inferior vena cava 702 through standard interventional techniques and may then be expanded within the vessel.

An introducer sheath 710 may be placed in a popliteal vein 708 that provides access to the patient’s venous vasculature proximal to the clot 602. The device 102 can then be inserted into the introducer sheath 710 and directed toward the clot 602 using standard interventional techniques. In some implementations, a longer introducer sheath or guiding catheter is first placed proximal to the clot 602 and then the device 102 is inserted through the sheath or guiding catheter. Any number of methods of navigating the device 102 to the clot 602 are contemplated. The catheter body 104 extends through the introducer sheath 710, through the popliteal vein 708, through the femoral vein 706, and to the proximal end of the clot 602 within the iliac vein 704.

In the implementation shown, the collection assembly 112 includes a filter assembly 712. The filter assembly 712 may include a series of meshes, such as mesh 714, that can separate the clot 602 from the blood following ingestion by the device 102. The meshes can have a pore size that may range from and including about 1 µm to and including about 500 µm, or about 200 µm. In some implementations, there may be multiple meshes that have different pore sizes for removing different constituent sizes. The filter assembly 712 may include a return tube 716 that connects to a port on the introducer sheath 710, which allows separated blood to be reintroduced into the patient. In alternative implementations, the return tube 716 may connect to a separate vascular access site. The filter assembly 712 beneficially prevents excessive blood loss from the patient by returning blood to the vasculature while permitting removal of clots or other similar material.

In the exemplary clinical application shown in FIG. 7, the device 102 is actuated by the user with a corresponding control, e.g., the throttle 418 of the handle assembly 106, and removes clot material and blood from the patient as described in FIGS. 3A-3F. After some or all of the clot 602 is removed, the device 102 and distal protection element 718 can be removed.

In other implementations, the device 102 can approach the clot 602 from above (i.e., the opposite direction) via an access site in the jugular vein. In such an implementation, the distal protection element 718 can optionally be configured to be located on the catheter body 104 or distal sleeve 120. Alternatively, the device 102 can be inserted through the distal protection element 718 by, for example, incorporating it into the introducer sheath 710 or guiding catheter.

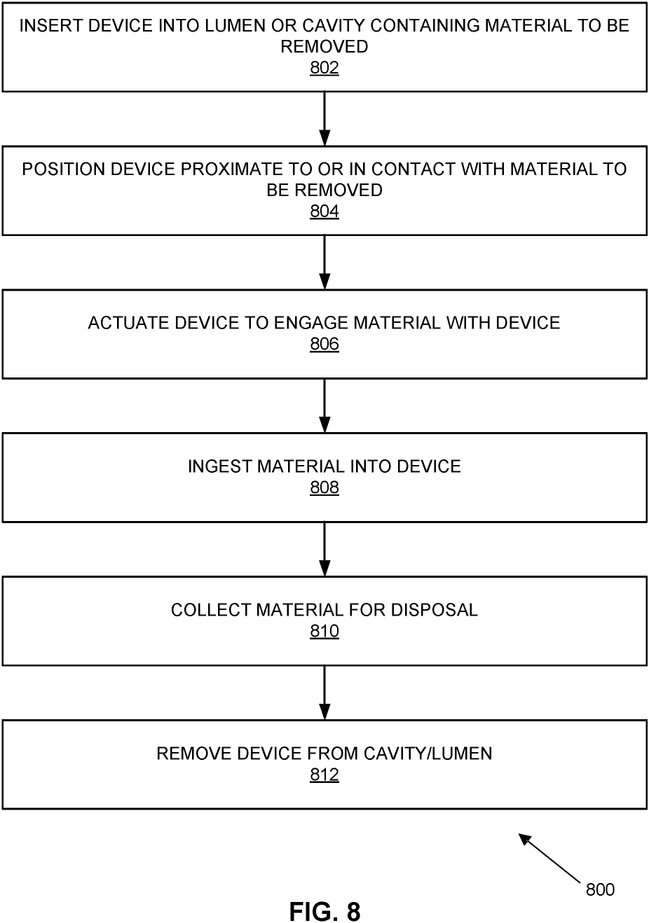

In FIG. 8, a method 800 for treating a patient to remove material from a lumen or cavity is provided. In step 802, a device according to this disclosure is inserted into the patient and positioned into a lumen or cavity containing material.

In step 804, the device can be advanced either proximate to or into contact with the material, depending on whether or not aspiration will be used to draw the material to the tissue engagement portion of the device.

In step 806, the device is actuated to initiate ingestion of the material into the device. In certain implementations, actuating the device may include actuating a pumping assembly of the device. As previously discussed, such actuation may engage and begin drawing the material into the catheter body of the device. For example, in implementations in which the device is brought into contact with the material in step 804, actuating the pumping assembly may cause the pumping assembly to mechanically engage the material. Actuating the pumping assembly may also create aspiration that draws the material into the pumping assembly instead of or in addition to the mechanical engagement and transportation. Finally, actuation of the device may also or alternatively include applying a separate aspiration source to draw material proximate or into contact with the tissue engagement portion of the device. The material may then be further captured by actuating the pumping assembly of the device, e.g., through mechanical engagement or suction/aspiration.

In step 808, the material is ingested into the device and thereby partially or fully removed from the lumen or cavity. by repositioning the tissue engagement portion or pumping assembly as needed. This may include advancing or retracting the device and/or rotating the tissue engagement portion or pumping assembly via the catheter hub.

In step 810, the removed blood and material is generally collected for disposal. Collection for disposal may include filtering material from other fluids (e.g., blood) to remove particulate and to permit reintroduction of fluids (e.g., blood) back into the patient.

In step 812, the device is removed from the lumen or cavity of the patient, substantially completing the clinical process.

In some implementations, the pumping assembly 110 may be longer than the exemplary implementation shown in FIGS. 3A-3F. For example, in the implementation shown in FIGS. 3A-3F, the stator helical lumen 210 has approximately one rotation which allows the rotor 116 to form a single closed cavity 224 within the stator 114. For purposes of this disclosure, such an arrangement is generally referred to as a single stage pump/single stage pumping assembly.

With reference to the dimensions shown in FIGS. 2C and 2D, in other implementations, additional stages may be added by increasing the length of the stator Lₛ and the number of rotations within the stator helical lumen 210 and/or by decreasing the pitch of the stator Pₛ. Such modifications can increase the number of closed cavities 224 formed by the rotor 116 and stator 114 during operation. Among other things, the additional stages of the pump may allow the pumping assembly 110 to create a higher head pressure as compared to single stage implementations.

With the foregoing in mind, in some implementations, the pumping assembly 110 includes at least a single stage; however, in other implementations, the pumping assembly 110 may include any suitable number of stages including, but not limited to, two (2), three (3), or up to five (5) stages. In yet other implementations, the pumping assembly 110 may include more than five (5) stages, e.g., up to one hundred 100 stages. In the latter implementations, the pumping assembly 110 may extend along a substantial portion up to the entire length of the catheter body 104. In still other implementations, the pumping assembly 110 may not include a full single stage such that the stator helical lumen 210 has less than one helical revolution.

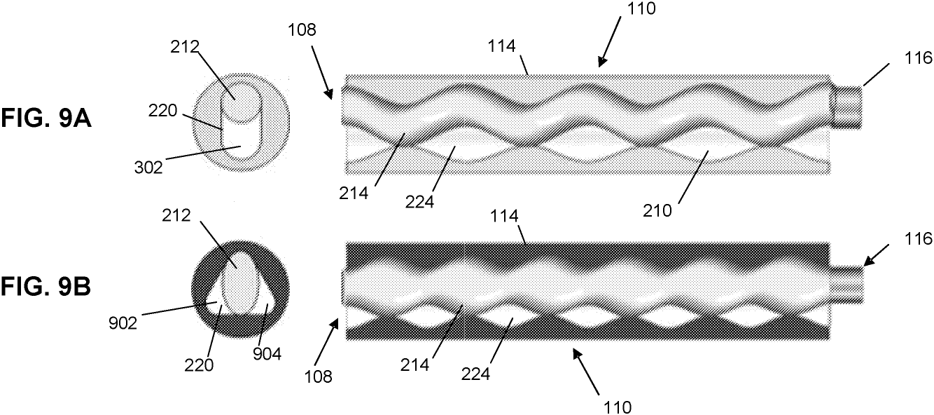

FIGS. 9A and 9B illustrate additional implementations of the present disclosure. In FIG. 9A, the stator helical lumen 210 and rotor helical section 214 create three (3) closed cavities. Among other things, stator/rotor configurations including more cavities may enable higher pressures and may allow for a reduced compression seal between the rotor 116 and stator 114 or a clearance gap such that the formed cavities are unsealed cavities.

FIG. 9A illustrates an example of a device in which the rotor distal tip 212 is generally circular and the stator helical lumen 210 approximates an obround slot 220 in the front view shown. In contrast, FIG. 9B illustrates an alternative implementation in which the profile of the rotor distal tip 212 has an ovate shape and the stator helical lumen 210 approximates a triangle slot 220 with rounded corners. Among other things, such a configuration can create multiple open cavities in the tissue engagement portion 108 as shown by the first open triangular cavity 902 and the second open triangular cavity 904. The implementation shown in FIG. 9B includes ten (10) closed cavities along its length while having a shorter pitch than the implementation shown in FIG. 9A.

More generally, the stator and rotor of implementations of this disclosure may be configured to form any number of cavities during operation. For example, in certain implementations, the stator and rotor may be configured to form from one and including a single cavity to and including about 200 cavities. Implementations including relatively high numbers of cavities may be facilitated by extending the stator 114 a substantial proportion up to the entire length of the catheter body 104 with the stator helical lumen 210 and rotor helical section 214 extending throughout. In other implementations, the rotor helical section 214 may be longer than the stator helical lumen 210 such that it extends out the proximal end of the stator 114 into the distal sleeve 120 or catheter body 104.

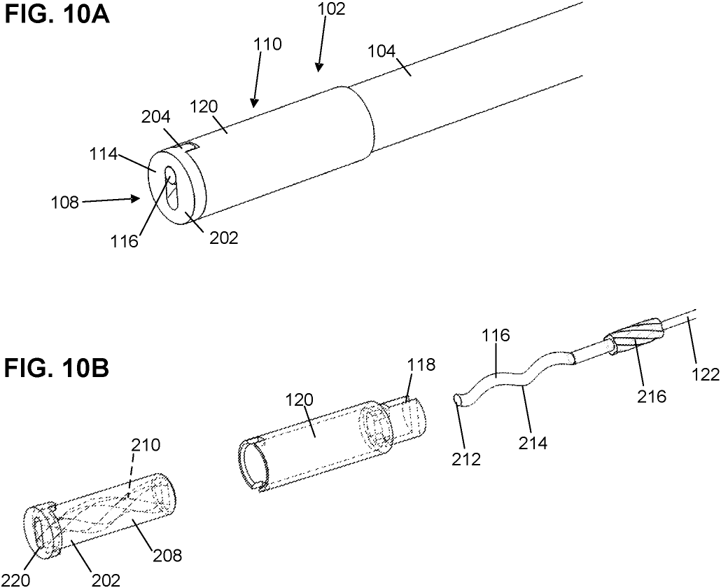

FIGS. 10A and 10B illustrate another alternative implementation of the device 102. More specifically, in the example of FIGS. 10A and 10B the distal sleeve 120 is generally straight and in-line with the axis of the catheter body 104. This is in contrast to some of the previously discussed implementations in which the device 102 included a distal bend. In one specific example of the implementation shown in FIGS. 10A and 10B, the length of the stator Lₛ may be about 20 mm long and the rotor 116 may include a short straight section between the rotor helical section 214 and the rotor hub 216 (shown in FIG. 10B). FIG. 10B also illustrates the rotor hub 216 as including spiral cuts that may propel ingested material into the catheter body 104 during operation/rotation of the rotor 116.

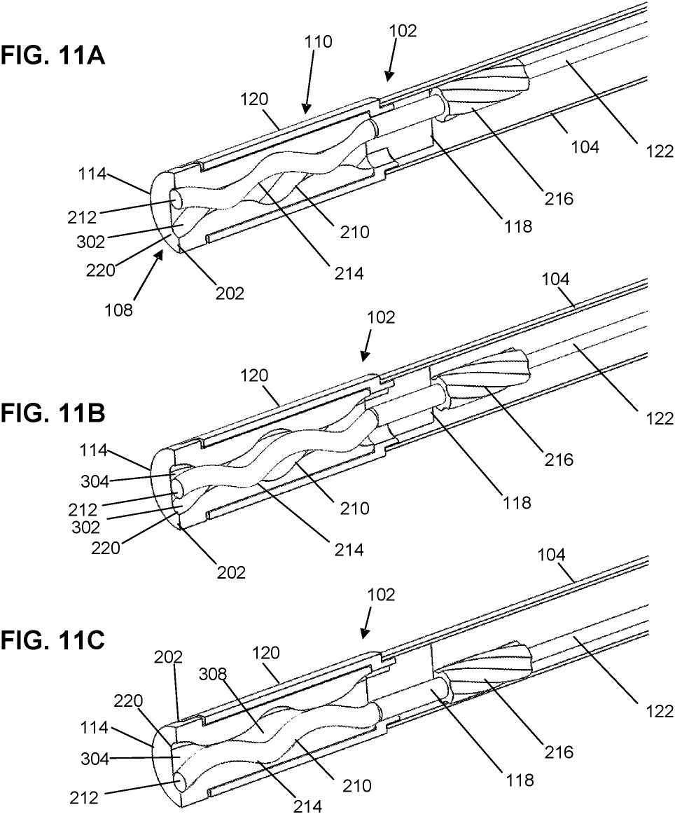

FIGS. 11A-11C, illustrate operation of the implementation shown in FIGS. 10A and 10B. As illustrated, the rotor hub 216 engages with the locating element 118, which is shown as an integral part of the distal sleeve 120. The locating element 118 feature axially locates the rotor 116 relative to the stator 114 such that the rotor distal tip 212 is flush with the end of the stator distal section 202 at the tissue engagement portion 108. In FIG. 11A, the pumping assembly 110 is in an initial configuration with the rotor distal tip 212 at the top of the slot 220. At the slot 220, the space between the rotor helical section 214 and the stator helical lumen 210 creates a first open cavity 302 which is open to the distal end of the device 102 allowing fluid and material to enter the first open cavity 302.

In FIG. 11B, the rotor 116 is rotated about one-quarter of a revolution relative to the configuration shown in FIG. 11A. As a result of the rotation, the rotor distal tip 212 has moved about half-way down the open slot 220. The opening to the first open cavity 302 is reduced as the cavity volume moves into the pumping assembly 110. A second open cavity 304 has opened and fluid or material can similarly enter this cavity. The first open cavity 302 and second open cavity 304 can be discrete fluid spaces which are either partially or entire sealed from one another.

In FIG. 11C, the rotor 116 is rotated another one-quarter of a revolution relative to the configuration shown in FIG. 11B. As a result, the rotor distal tip 212 is now at the bottom of the slot 220. The first closed cavity 308 is no longer substantially connected to the distal end of the device 102 and has moved entirely within the stator 114. The second open cavity 304 is now fully open to the distal end of the pumping assembly 110 and fluid or material continue to be ingested into the cavity. As the rotor 116 continues to rotate, the first closed cavity 308 will be pumped into the catheter body 104 and then the second open cavity 304 will likewise be pumped into the catheter body 104. This process continues with new cavities being formed at the stator distal section 202 which are then ingested into the stator 114 and pumped into the catheter body 104. The assembly therefore works like a progressive cavity pump, ingesting material into the stator 114 and expelling it into the catheter body 104.

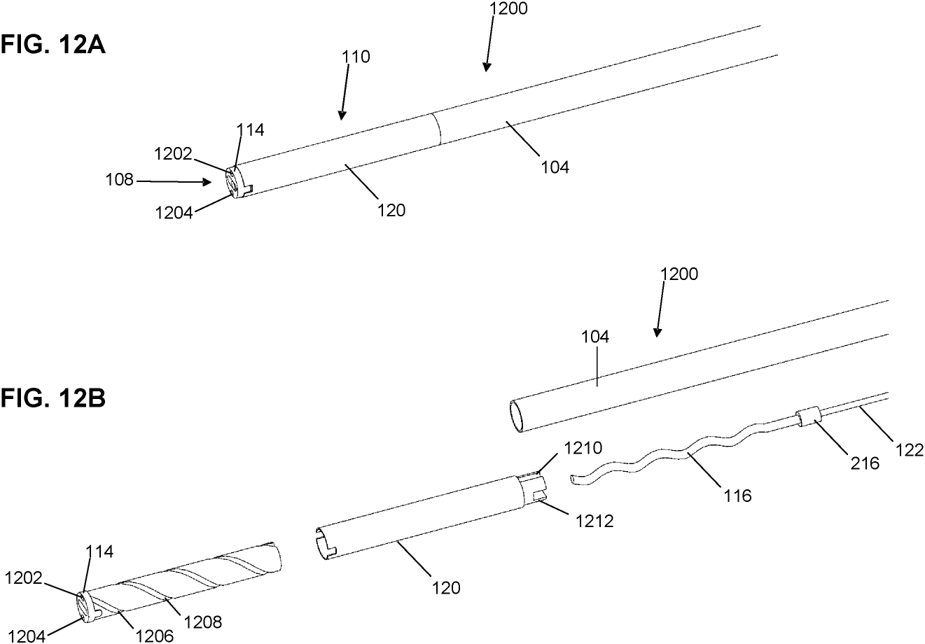

In FIGS. 12A and 12B, an implementation a device 1200 of the device 102 is shown that includes one or more additional channels (namely, an aspiration inlet 1202 and an infusion outlet 1204) in the tissue engagement portion 108 to facilitate infusion and/or aspiration. More specifically and as compared to previous implementations, the stator distal section 202 is modified to include the one or more additional channels, which are fluidly connected to the catheter body 104.

In FIG. 12A, an aspiration inlet 1202, which can remove material, and an infusion outlet 1204, which can deliver material, are shown. In FIG. 12B, the implementation of FIG. 12A is shown in more detail in an exploded view. As shown, the stator 114 has channels along its outer surface that allow for direct fluid communication between the distal end of the device 102 and the catheter body 104. In some implementations, these channels are lumens that run through the stator 114 or distal sleeve 120. An aspiration channel 1206 connects the aspiration inlet 1202 and an aspiration outlet 1210 in the distal sleeve 120. An infusion channel 1208 connects the infusion outlet 1204 with an infusion inlet 1212 in the distal sleeve 120. The aspiration channel 1206 can be used to apply suction by a separate connected aspiration source such as a pump or a syringe which can mobilize and hold the clot to the tissue engagement portion 108 while the pump assembly disrupts and ingests the clot. For example, an aspiration syringe can be connected to the outlet tube 132 which is fluidly connected to the aspiration inlet 1202 through the aspiration channels 1206. When vacuum pressure is applied via the syringe, fluid can flow through the aspiration inlet 1202 which can allow the device 102 to draw material to the device or hold it there while the rest of the tissue engagement portion 108 and the pumping assembly 110 removes the clot. In this manner, the aspiration inlet 1202 can be an integral part of the tissue engagement portion 108.

Likewise, the infusion inlet 1212 can be connected to a tube that allows the user to inject materials to the distal end of the device 102 through the infusion channel 1208. For example, contrast dye could be injected to the front of the device 102. Alternatively, drugs or any other clinically suitable material can be injected. In other implementations, saline can be injected through the infusion outlet 1204. This may beneficially provide a low-viscosity fluid for the pumping assembly 110 to assist with clot extraction and reduce blood loss. Alternatively, infused saline may be used to pressurize the vessel such that the vessel does not collapse due to negative pressure created by the pumping assembly 110 or separate aspiration source. Infused saline may also be used to assist the tissue engagement portion 108 ingesting the material. The pumping assembly 110 may perform better when thicker material is mixed with viscous fluids such as water, saline, or blood. The infused fluid may also be sprayed at high velocities to provide disruption of the thicker materials at the tissue engagement portion 108 and thereby improve the ingesting performance. In some implementations, the infused fluid may include saline and dextrose that can be used to flush out blood out of the vessel in order to improve clot visualization with technologies such as optical coherence tomography (OCT).

Extrusions or tubing within the catheter body 104 can be used to fluidly connect the aspiration outlet 1210 and infusion inlet 1212 to the handle assembly 106. In the illustrated implementation, the channels 1206 and 1208 are formed between the stator 114 and the distal sleeve 120 and are wrapped helically around the stator 114 to avoid the stator helical lumen 210. In other implementations, the channels may be straight or in another non-helical pattern. In still other implementations, both channels can be aspiration channels 1206 or infusion channels 1208, or the device 102 can have only one channel or more than two channels. In still other implementations, the channels may be used for the insertion of guidewires or other devices that are useful in standard catheter procedures. For example, a guidewire can be inserted through one of the channels and used for delivering the device to the site within the patient lumen or cavity.

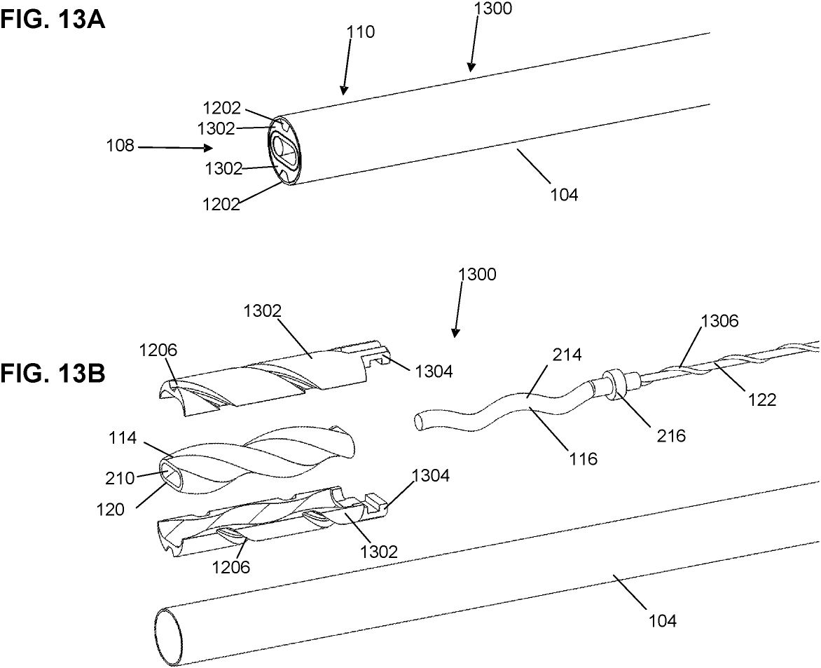

FIGS. 13A-13B illustrate an alternate implementation of a device 1300 according to this disclosure. More specifically and in contrast to previously illustrated implementations, the device 1300 is shown with an even wall-thickness stator 114. In FIG. 13A, the distal end of the device 102 is shown which includes two stator supports 1302. The catheter body 104 extends over the entire distal end of the device 102 and contains the stator supports 1302 without a distal sleeve 120.

In FIG. 13B, the device 1300 of FIG. 13A is shown in more detail in an exploded view. As shown, the stator 114 has a similar stator helical lumen 210 and slot 220 as in previous implementations. However, in contrast to previously discussed implementations, the external profile of the stator 114 is not a cylinder. Rather, the outer profile of the stator 114 matches the stator helical lumen 210 such that an approximately uniform wall thickness is maintained throughout the stator 114. The wall-thickness may vary depending on the overall size of the catheter and clinical use; however, in certain example implementations the wall thickness may be from and including about 0.1 mm to and including about 10 mm, from and including about 0.2 mm to and including about 1 mm, or about 0.4 mm.

The stator supports 1302 generally include an internal surface that matches the exterior profile of the stator 114. When the rotor 116 spins within the stator 114, the stator helical lumen 210 is compressed between the rotor helical section 214 and the stator support 1302. Since the stator 114 has an approximately uniform wall-thickness, the amount of compression and sealing performance can be maintained along the entire helical line of contact. However, this disclosure contemplates that other outer profiles of the stator 114 are possible and, in particular, implementations in which the stator 114 may not have a uniform wall thickness.

The device 1300 further includes aspiration channels 1206 which run on the outer surface of the stator supports 1302. This configuration of the aspiration channels 1206 may be beneficial since they exist in the stator support 1302 and therefore would not affect sealing of the stator 114. In certain implementations, the stator support 1302 material may be stiffer than the stator 114 material, e.g., the stator support 1302 may be formed form a hard plastic or metal while the stator 114 may be formed from an elastomeric material. Alternatively, each of the stator supports 1302 may be formed of an elastomer, such as silicone or polyurethane, which is stiffer than the stator 114 material.

The stator supports 1302 include a positioning feature 1304 for interacting with the rotor hub 216. In the illustrated implementation, the positioning features 1304 provide both a distal and proximal locating element 118 for the rotor hub 216. Such a positioning feature 1304 can be incorporated in any implementation described herein. The implementation of FIG. 13B further includes an auger feature 1306 extending along the torque member 122. Among other things, the auger feature 1306 can assist in urging fluid and material proximally within the catheter body 104 and reduce clogging of the lumen of the catheter body 104. The auger feature 1306 can be a separate component surrounding the torque member 122 or can be integrally formed with the torque member 122.

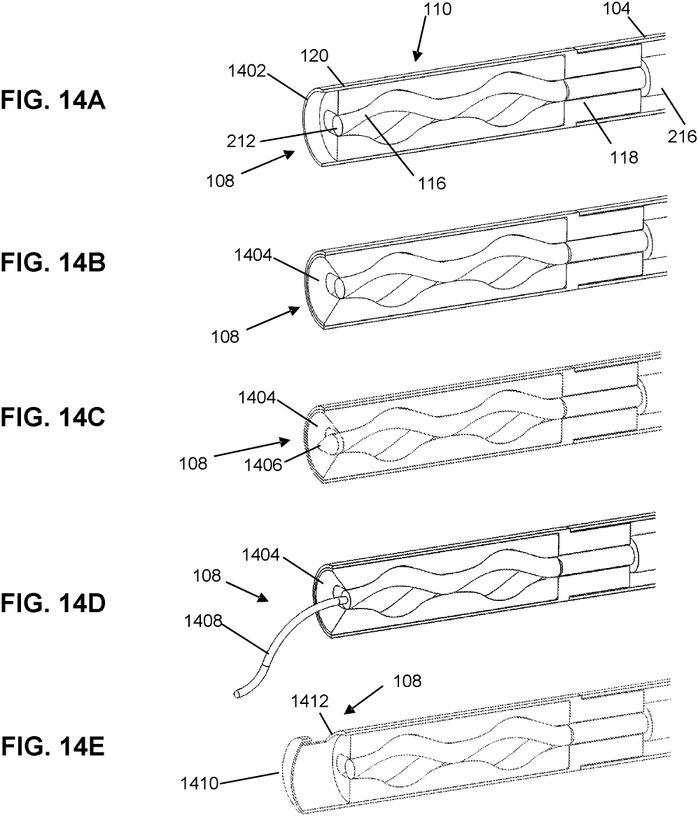

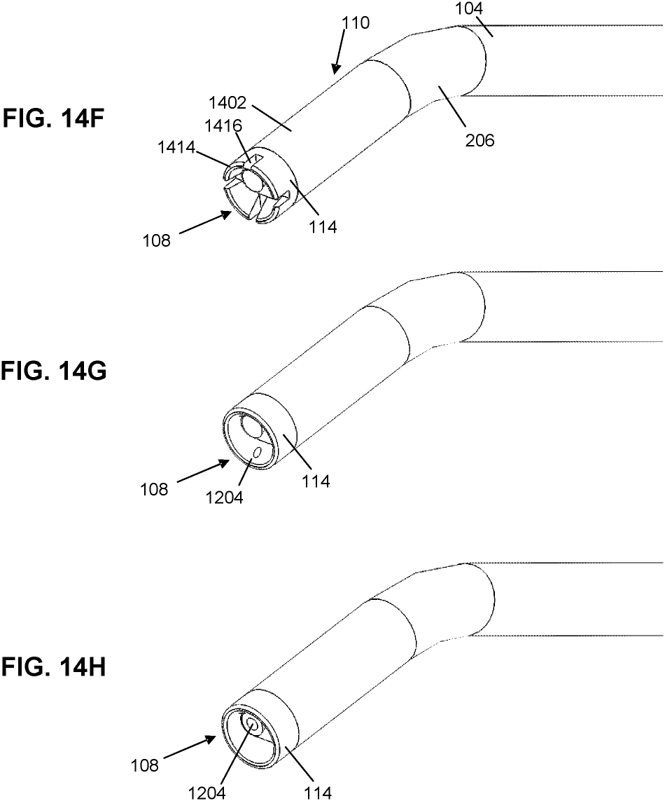

FIGS. 14A-14H illustrates several alternative implementations of the tissue engagement portion 108 of devices according to this disclosure. In FIG. 14A, the tissue engagement portion 108 includes an extension feature 1402 that extends beyond the end of the stator 114. The rotor distal tip 212 is therefore inset which may beneficially prevent injury to vessels, among other things. The length of the extension feature 1402 may vary; however, in certain example implementations, the extension feature 1402 may be from and including about 0.1 mm to and including about 100 mm, from and including about 1 mm to and including about 10 mm, or about 2 mm. The extension feature 1402 can be formed by the distal sleeve 120 as shown. Alternatively, the extension feature 1402 may be formed by the catheter body 104 or may be a separate component coupled to the distal sleeve 120. Although illustrated as being substantially cylindrical, in other implementations, the extension feature 1402 can end with a beveled tip or be non-cylindrical. In other implementations, the extension feature 1402 can be expandable and include braids, laser cut components, or similar structures that create a cage around the rotor distal tip 212 and stator distal section 202 to provide a barrier for vessels or other tissues from being ingested into the pumping assembly 110. In still other implementations, the extension feature 1402 can be slidably or rotationally adjustable relative to the pumping assembly 110. For example, an outer tube over the device 102 may be used to increase or decrease the amount that the distal face of the stator 114 is recessed within the extension feature 1402. In yet other implementations, the pumping assembly 110 can be positioned anywhere along the length of the catheter body 104 such that the extension feature 1402 is simply the portion of the catheter body 104 which extends distal to the pumping assembly 110. For example, the pumping assembly 110 can be positioned within the catheter body 104 half-way along the length of the catheter body 104.

FIG. 14B illustrates another implementation of the device 102 that includes a funnel feature 1404 on the tissue engagement portion 108. Among other things, the funnel feature 1404 can help guide fluid and material into the stator helical lumen 210 or can alternatively provide a suction cup feature for securing material to the tissue engagement portion 108 during ingestion.

In FIG. 14C, the rotor distal tip 212 includes a rotor tip extension 1406 that extends forward into the funnel feature 1404. The rotor tip extension 1406 can assist the tissue engagement portion 108 in disrupting and ingesting material into the stator 114. The rotor tip extension 1406 can have a pointed geometry as shown or can be any number of other shapes and configurations such as an auger shape or paddle shape. In some implementations, the rotor tip extension 1406 can remain within the extension feature 1402 or can extend beyond the distal end of the device 102. In some implementations, the rotor tip extension 1406 can be advanced forward or retracted by the user. For example, the rotor 116 can be advanced forward to extend the rotor tip extension 1406 during certain parts of the operation to assist in disrupting the clot and removing wall adherent clot. During this operation the handle assembly 106 may rotate the rotor 116 at a slower speed to prevent vessel injury. The rotor 116 can then be retracted such that the rotor distal tip 212 is flush with the end of the stator 114 and risk of vessel injury is minimized which can allow for faster motor rotational speeds.

In FIG. 14D, the rotor 116 has a helical lumen that runs through the length of the rotor 116 allowing passage of an extension wire 1408 or similar elongate structure through the rotor 116. The extension wire 1408 can be advanced out of the end of the rotor 116 to create an advanceable rotor tip extension 1406 in the tissue engagement portion 108. The extension wire 1408 may include a predetermined shape such as a curve or hook which is configured to contact the vessel wall and disrupt the clot. The motor speed of the handle assembly 106 can be controlled depending on whether the extension wire 1408 is extended or not. In some implementations, the extension wire 1408 is not adjustable and is an integral part of the rotor 116. Alternatively, the extension wire 1408 may be used as a guidewire for navigation and delivery of the device 102 to the target site.

In FIG. 14E, the distal sleeve 120 includes a capped end 1410 that may beneficially prevent the tissue engagement portion 108 from ingesting vessel walls, valves, or other tissues which are not intended to be removed by the device 102. The distal sleeve 120 further includes a side cut 1412 that may direct material into the tissue engagement portion 108 from the side. This may be particularly useful for materials that exist on the lumen walls of the vessel. In this implementation, the side cut 1412 may be rotated circumferentially using a mechanism like the catheter hub 124.

In FIG. 14F, the tissue engagement portion 108 includes a series of thrombus standoffs (e.g., standoff 1414) that protrude from the distal end of the stator 114. The gaps between adj acent thrombus standoffs can form one or more fluid inlets (e.g., fluid inlet 1416). The fluid inlets can allow low viscosity fluids like blood still enter the pumping assembly 110 while thicker material is being ingested. This may beneficially enable the pumping assembly 110 to ingest a mixture of blood and thrombus rather than thrombus only which may improve the performance of the pumping assembly 110.

In FIG. 14G, the tissue engagement portion 108 includes an infusion outlet 1204 in the stator 114. The infusion outlet 1204 can be used for the injection of fluid like saline that can provide improved performance of the pumping assembly 110. Injected fluid can ensure that the pumping assembly 110 has an optimal mixture of low and high viscosity materials. In addition, the infusion outlet 1204 can be directed at an angle and used to spray thick materials at high speeds. This can beneficially break up the thick materials, such as thrombi, before ingestion by the pumping assembly 110. The infusion outlets 1204 can be directed at an angle or perpendicular to the end of the stator 114.

In FIG. 14H, the tissue engagement portion 108 includes an infusion outlet 1204 through the rotor 116. For example, the rotor 116 can have a lumen running through its center and connected to a hollow torque member such as a tube or torque coil. The injected fluid can assist in the performance of the pumping assembly 110. Alternatively, the injected fluid can include contrast media, fluids with active pharmaceutical ingredients (APIs), or any number of other desired fluids.

Any combinations of the features or elements in the foregoing implementations may be used alone or in combination with other implementations discussed herein. For example, the rotor tip extension 1406 may exist within the capped end 1410 such that it disrupts clot before ingesting it in the pumping assembly 110 but has limited risk of vessel damage since the rotor tip extension 1406 is contained within the distal sleeve 120.

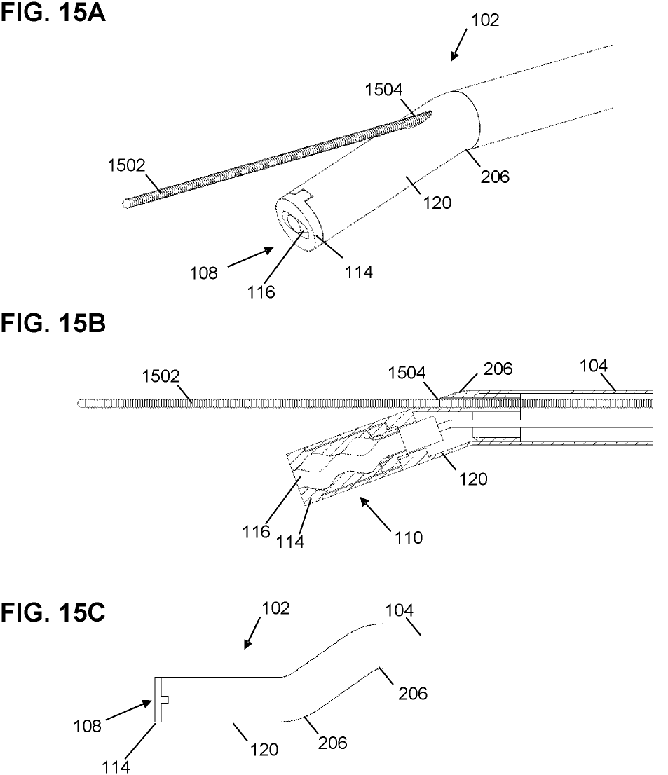

FIGS. 15A and 15B illustrate an implementation of the device 102 including a guidewire 1502. In general, the device 102 is delivered through a patient’s vasculature to material to be removed. Certain catheterization techniques involve advancing devices over guidewires or smaller catheters. In the illustrated implementation of FIGS. 15A and 15B, the guidewire 1502 is inserted through an entry hole 1504 near the distal bend 206 of the distal sleeve 120. The entry hole 1504 is generally sized to accommodate any suitable guidewire or microcatheter; however, in at least certain implementations, the entry hole 1504 may have a diameter from and including about 0.008 in to and including about 0.080 in, or about 0.035 in. This beneficially allows the guidewire 1502 to be navigated and placed in the correct location within the patient’s vasculature and then the device 102 can be advanced over the guidewire 1502 to the target site. With the distal bend 206, the pumping assembly 110 is offset from the axis of the catheter body 104 and the guidewire 1502 such that the lumen of the pumping assembly 110 does not need to be clear of the guidewire 1502 in order for the device 102 to track over the guidewire 1502. In some implementations, an additional exit hole can exist on the catheter body 104 or the distal sleeve 120 which allows for a rapid exchange of the catheter. Once the guidewire 1502 is in situ, the proximal end of the guidewire 1502 can be fed through the entry hole 1504 and then out of an exit hole in the catheter body 104. In this manner, the guidewire 1502 does not need to be excessively long.

This disclosure contemplates other methods of navigating the device 102 over a guidewire 1502. In some implementations, the rotor 116 can be removed from the stator 114 such that a guidewire 1502 can go through the lumen created by the stator helical lumen 210. Once at the target site, the guidewire 1502 can be removed and the rotor 116 can be inserted into the stator 114. In other implementations, the rotor 116 and/or torque member 122 can include a hollow lumen through their center that can allow passage of a guidewire. In such an implementation, the proximal end of the guidewire 1502 can be inserted through the rotor 116 and the torque member 122 to enable navigation of the device 102 and the guidewire 1502 can then be removed. In still other implementations, the guidewire 1502 can be back loaded into channels 1206 such that the device 102 tracks over the guidewire 1502 which helically wraps around the stator 114 within the distal sleeve 120.

In FIG. 15C, an implementation of the device 102 is shown with multiple distal bends 206. The multiple distal bends 206 allow the axis of the stator 114 to remain relatively parallel to the axis of the catheter body 104 but offset such that rotation of the catheter body 104 moves the tissue engagement portion 108 within the vessel. Such an implementation may enable improved reach of the device 102 within a large vessel without adversely directing the tissue engagement portion 108 toward the vessel wall. Any number of other suitable bends and orientations of the stator 114 relative to the catheter body 104 are contemplated.

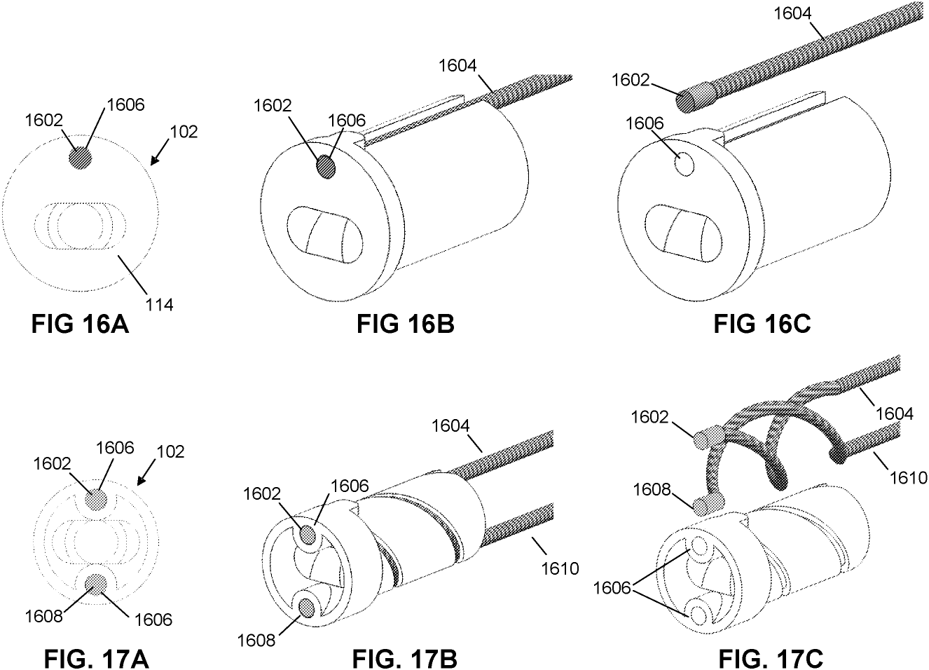

In FIGS. 16A-16C, an implementation of the device 102 is shown with a visualization assembly 1602 at the distal end of the stator 114. A stator opening 1606 may hold and/or orient the visualization assembly 1602 facing forward and may be offset from the stator helical lumen 210 so that the increased catheter size of the device 102 with the visualization assembly 1602 is minimized. Additionally, the visualization cable 1604 can be predominately straight as it travels along the length of the stator 114. In some implementations, the visualization assembly 1602 can be advanced forward and backward or rotated relative to the stator 114 to change perspective. The visualization assembly 1602 can be a camera such as a CMOS or CCD imaging device that provides visual feedback to the user about the tissue structures at the distal end of the device 102 or about the clot 602 in the blood vessel 604. Alternatively, the visualization assembly 1602 can be an intravascular ultrasound (IVUS) imaging probe or catheter. Alternatively, the visualization assembly 1602 can be an optical coherence tomography (OCT) element. For example, the OCT element may be a guidewire that can be advanced from the end of the device 102. The head of the visualization assembly 1602 can have a diameter of from and including about 0.25 mm to and including about 3.0 mm, from and including about 0.5 mm to and including about 2.0 mm, or about 1.0 mm. Alternatively, the head of the visualization assembly 1602 can be a rectangular profile.

The head of the visualization assembly 1602 can be connected with a visualization cable 1604 that runs inside of the catheter body 104 and out of the patient. The visualization assembly 1602 can include a display such as an LCD or LED screen to provide visual feedback to the user. In some implementations, the user may watch the display and accordingly advance or turn the device 102 such that distal end of the catheter is directed toward the desired tissue such as clot. The visual feedback can assist the user in navigating the end of the device away from or avoiding activating the device 102 near the vessel wall, vein valves, or other important tissue structures. Additionally, the visual feedback can indicate to the user when the vessel is collapsing from negative pressure or too high of aspiration flow generated by the device 102.

In some implementations, the information provided by the visualization assembly 1602 can be used by software within or executed by a computing device in communication with the device 102. For example, the software could disable the device 102 if the software detects that the distal end of the device 102 is too close to a tissue structure such as the vessel wall or vein valve. In other implementations, the software can create alarms or alerts to the user in such situations. In some implementations, the software can assist the user in navigating to the material by providing audible or visual indicators. In other implementations, aspects of the device 102 can be controlled by automatically such as the orientation of the distal end, the advancement of the distal end in the vessel, and the activation of the pumping assembly 110. In this manner, the visualization assembly 1602 can enable more automated aspects of the procedure by providing feedback to the software. In some implementations, the visualization assembly 1602 can provide feedback to the user about where clot 602 is within the vessel 604. For example, if the user moves the device 102 through the vessel 604, some clot 602 may be removed but some may remain, particularly wall-adhered clot 602. The visualization assembly 1602 can indicate to the user where to direct the tissue engagement portion 108 and thereby provide a more targeted clot 602 removal. In some implementations, the visualization assembly 1602 can be advanced through the vessel 604 and then withdrawn and a 2D or 3D representation of the vessel 604 can be displayed to the user. In some implementations, the visualization assembly 1602 is not at the very distal tip of the device 102 but rather is along the side of the pumping assembly 110 or within the catheter body 104.

In FIGS. 17A-17C, an implementation of the device 102 is shown with a first visualization assembly 1602 and a second visualization assembly 1608 which wrap helically around the stator 114. By wrapping the first visualization cable 1604 and the second visualization cable 1610 around the stator 114, the overall size profile of the device 102 does not increase or increases minimally. In some implementations, the first visualization assembly 1602 is a camera and the second visualization assembly 1608 is an IVUS probe. In other implementations, both assemblies can be cameras and provide stereoscopic vision. In other implementations, the visualization assemblies can be directed at different angles or provide different focus depths. The second visualization assembly 1608 does not necessarily need to be an imaging element. It can also be a light source for the first visualization assembly 1602 or for external visualization through the patient’s skin to enable direct visualization of the device 102. In other implementations, the second visualization assembly 1608 can be a tube for delivery of materials to the distal end of the device that aide in visualization. For example, the second visualization assembly 1608 can deliver saline or other clear fluids that are transparent for the first visualization assembly 1602 and can provide positive pressure within the vessel that maintain the patency of the vessel and prevent collapse. In still other implementations, the visualization assembly 1602 does not necessarily require a visualization cable 1604 and can include communication electronics for transmitting information to a display or to the software of the device 102 using Wi-Fi, Bluetooth, or any other suitable method.

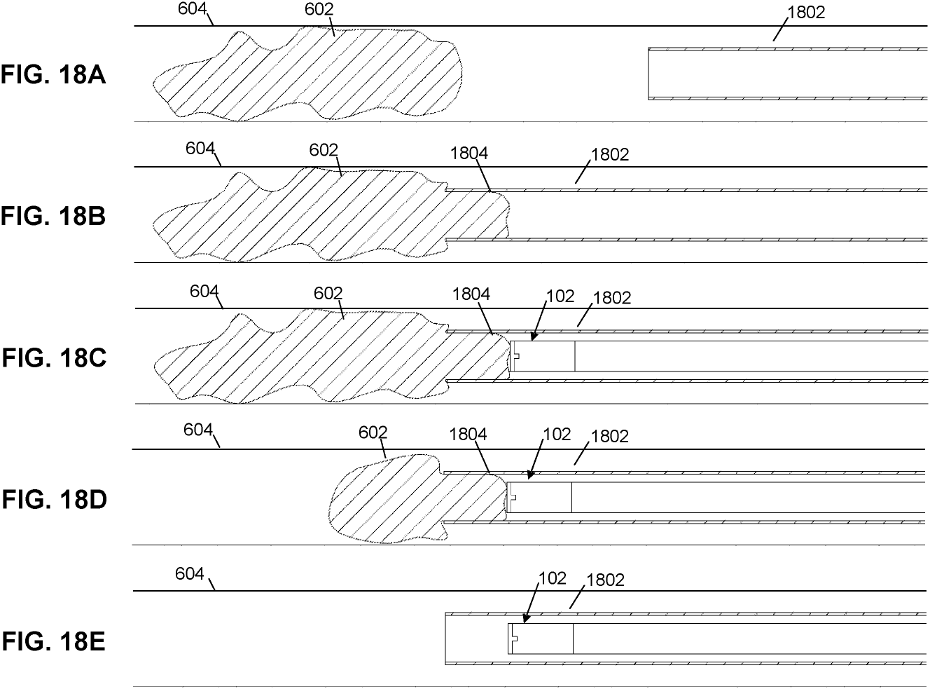

In FIGS. 18A-18E, an implementation of the device 102 is shown with an aspiration catheter 1802. The aspiration catheter 1802 is inside of a blood vessel 604 that has material to be removed, in this case a clot 602, which obstructs blood flow.

In FIG. 18B, the aspiration catheter 1802 is advanced to the clot 602 and an aspiration source is connected to the catheter which engages the clot 602. As shown, the clot 602 has a corked portion 1804 which is within the aspiration catheter 1802. In this configuration, the vacuum level may be intentionally reduced to prevent ingestion of the clot 602. Alternatively, the situation illustrated in FIG. 18B may result from the maximum level of vacuum of the aspiration catheter 1802 being insufficient to fully ingest the clot 602.

In FIG. 18C, a version of the device 102 is inserted through the lumen of the aspiration catheter 1802 and advanced to the clot 602 to facilitate further ingestion of the clot 602. As described throughout this disclosure, the device 102 can be made to abut or be disposed proximal the clot 602 and then activated to ingest the clot 602 into the device 102, e.g., using one or both of mechanical engagement with the clot 602 and aspiration. For example, in FIG. 18D, the clot 602 has been partly ingested into the device 102 and in FIG. 18E, the clot 602 has been completely removed from the vessel 604.

In some implementations, the device 102 can be the aspiration source for the aspiration catheter 1802 and can create the vacuum level to engage the clot 602 onto the tip of the aspiration catheter 1802. In such implementations, the pumping assembly 110 can be disposed within the aspiration catheter 1802 a distance away from the distal end from and including about 0 cm to and including about 100 cm, or from and including about 0.1 cm to and including about 20 cm or about 0.5 cm. When the device 102 is activated, aspiration is formed within the aspiration catheter 1802 which pulls clot 602 into the aspiration catheter 1802 and up to the tissue engagement portion 108. The clot 602 is then ingested into the pumping assembly 110 and discharged into the lumen of the catheter body 104 or the lumen of the aspiration catheter 1802. The position of pumping assembly 110 can adjust as needed and in some implementations can be advanced beyond the end of the aspiration catheter 1802. In this manner, the user can adjust the location of the tissue engagement portion 108 depending on the clot type, stage of the procedure, or any other reason. In some implementations, the aspiration catheter 1802 can be close fitting to the outer diameter of the device 102 such that the catheter forms a slidable outer tube over the device 102 that can engage the clot 602 and can also push clot off the tip of the device 102 to prevent or remove clogging of the pumping assembly 110. In other implementations, the outer diameter of the device 102 can be smaller than the inner lumen of the aspiration catheter 1802 such that fluid can go around the lumen of the device 102. In some implementations, the catheter body 104 is replaced by a control wire that can advance, retract, or turn the pumping assembly 110. The lumen of the aspiration catheter 1802 may therefore be maximized along its length and deliverability of the device 102 through the aspiration catheter 1802 may be improved.

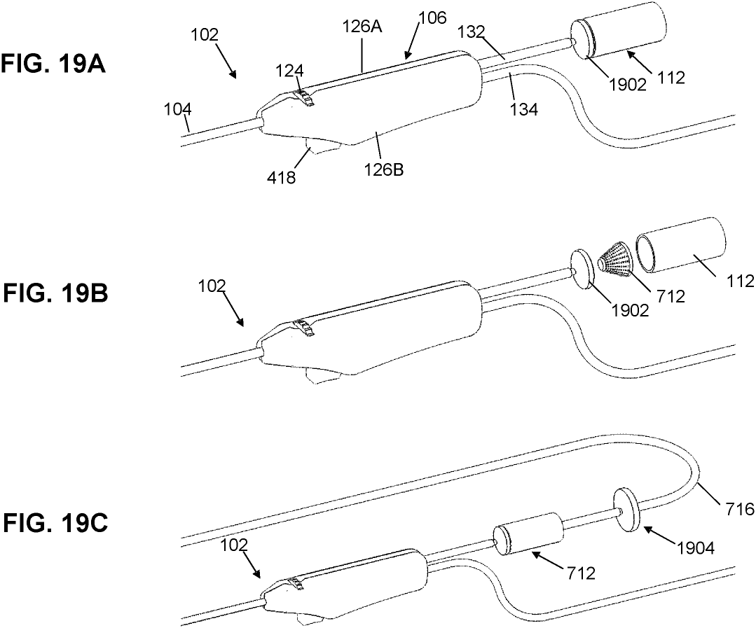

In FIGS. 19A-19C, implementations of the device 102 are shown that include filter assemblies. In FIG. 19A, an alternative implementation of the handle assembly 106 is shown with a catheter hub 124, a throttle 418 and two handle shells 126A, 126B. The outlet tube 132 is connected to a collection assembly 112 with a filter assembly 712 and a collection cap 1902.

In FIG. 19B, the same device 102 is shown with the collection assembly 112 in an exploded state. During operation, material removed using the device 102 enters into the collection assembly 112 and is then filtered through the filter assembly 712. By doing so, solid or semi-solid material (e.g., thrombus material) is maintained in the front area of the collection chamber and lower viscosity material travels into and is separated into the rear area of the collection assembly 112. In certain implementations, the collection assembly 112 may be at least partially transparent to provide a visual indication of operation of the device 102 and corresponding capture of material within the collection assembly 112. In some implementations, the collection cap 1902 can further include a separate port for infusion that allows the user to inject a fluid (e.g., saline) such that the material in the front area of the collection assembly 112 can be better visualized. In addition, the filtered blood can be stored for reintroduction to the patient if necessary. In some implementations, the collection assembly 112 can be integral to the handle assembly 106 such that the outlet tube 132 may be omitted.

In FIG. 19C, the device 102 includes a series of filters for separating solid or semi-solid material from blood or other fluids and a return tube 716 for reintroduction of fluid into the patient. In one specific implementation, a first filter assembly 712 can have a pore size from and including about 25 um to and including about 1000 um, from and including about 100 um to and including about 400 um, or about 200 um. In the same implementation, the second filter assembly 1904 may have a pore size from and including about 5 um to and including about 100 um, or about 40 um. More generally, however, any number of additional filters and filter pore sizes can be implemented. Also, the return tube 716 can feed blood back to the patient through any number of access points such as the introducer sheath, a separate access site, or through the catheter body 104.

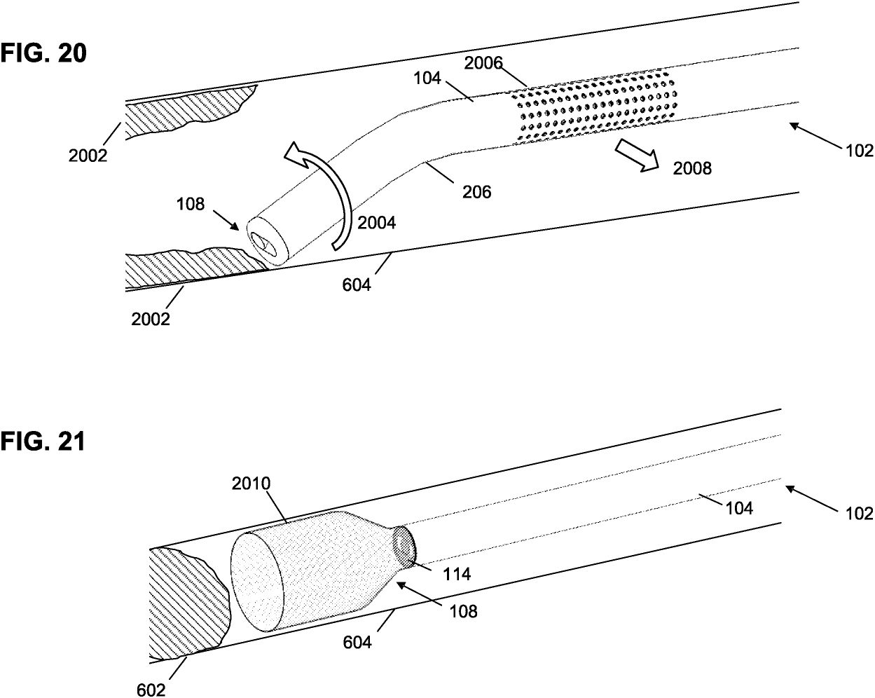

In FIG. 20, the device 102 is shown removing wall adherent material 2002. In certain disease states, material is adhered to the wall of a vessel 604. The material can include certain thrombus disease like DVT and can also include the plaque found in peripheral arterial disease and coronary arterial disease. The wall adherent material 2002 may be difficult to remove with a straight catheter body 104. In this implementation, the catheter body 104 includes a distal bend 206 and can be positioned using rotation 2004 of the catheter body 104. Such a device 102 can advantageously access the entire circumference of the vessel 604.

As an alternative or supplement to the proximal filtration and separation systems previously discussed, the device 102 may also include a catheter filter 2006 along the catheter body 104. The catheter filter 2006 can include a series of holes in the catheter body 104 as shown or can alternatively include a separate filter material. As blood and material is discharged from the pumping assembly 110 and pushed through the catheter body 104, the blood can be filtered from the material and can reenter the vessel 604 through the filtered blood return 2008. This may advantageously reduce the amount of removed blood. The catheter filter 2006 may include a variety of pore sizes and lengths.

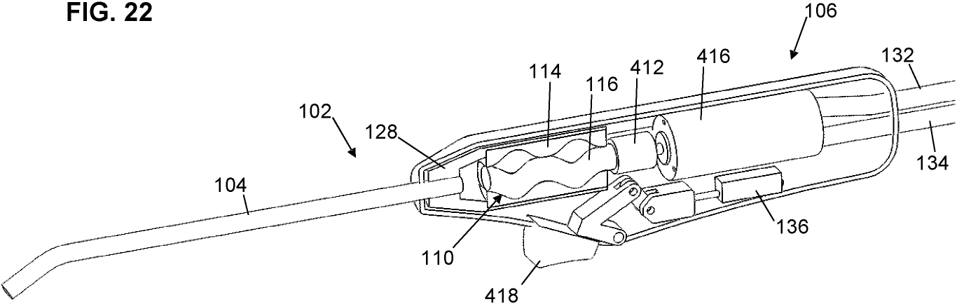

In FIG. 21 an alternative implementation of the device 102 is illustrated in which the tissue engagement portion 108 includes an expandable funnel 2010. The expandable funnel 2010 can be collapsed in a delivery sheath during introduction into the vessel 604. Once deployed, the expandable funnel 2010 can open in the vessel 604 and provide partial or complete flow arrest in the vessel 604. This may advantageously assist the tissue engagement portion 108 in pulling the material toward the pumping assembly 110 and can reduce the likelihood of particulate flowing downstream of the treatment site. In certain implementations, the expandable funnel 2010 can be comprised of a braided wire or a laser cut tube and may additionally include membranes such as silicone or PTFE that make the expandable funnel 2010 impermeable or semi-impermeable.

FIG. 22 illustrates an alternative implementation of the device 102 in which the pumping assembly 110 is disposed within the handle assembly 106. More specifically, the pumping assembly 110 shown is a progressive cavity pump which includes a stator 114 and rotor 116 and the operation of which is described herein. The handle assembly 106 has a motor 416 which is rotationally connected to a motor adaptor 412 and which is then rotationally connected to the rotor 116. A manifold 128 fluidically connects the inlet of the pumping assembly 110 to the catheter body 104 such that operation of the pumping assembly 110 withdraws fluid from the catheter body 104. The distal tip of the catheter body 104 can be open and therefore perform like a traditional aspiration catheter. Beneficially, such an implementation may not require a separate piece of equipment and the vacuum is generated closer to the tip of the catheter resulting in fewer line losses and greater dynamic control by the user. In the implementation shown, an outlet tube 132 evacuates the fluid to a waste container and an electrical cable 134 provides power for the device 102. In other implementations, a waste container may be incorporated into the handle assembly 106 as well as a battery such that no additional cables or lines extend from the device 102. In some implementations, the distal tip of the catheter body 104 may still include an additional pumping assembly 110 as described in other implementations. These two pumping assemblies 110 may be rotationally connected via a torque member 122 or may rotate independently. In other implementations, the distal tip of the catheter body 104 may include other maceration or tissue disruption elements such as screws, augers, or morcellators. Other types of pumps in the handle assembly 106 are contemplated such as piston pumps, diaphragm pumps, peristaltic pumps, centrifugal pumps, screw pumps, rotary gear pumps, or any other suitable pumping mechanism.

The following is a discussion of various alternative or supplemental features that may be included in implementations of this disclosure. Unless specifically noted, it should be assumed that any of the alternatives discussed in the following section may be incorporated into or applied to any implementations included in this disclosure or otherwise encompassed by its scope.

In some implementations, the rotor 116 may have a variety of coatings or textures along its rotor helical section 214. The coatings may be lubricious coatings such as hydrophilic or hydrophobic coatings or metal plating. Alternatively, the outer surface may be textured or coated to provide additional friction that pulls the clot into the stator 114. In still other implementations, the rotor helical section 214 may include an elastomeric coating on its outer surface so that it becomes the sealing member and the stator 114 can therefore be comprised of a harder material like a thermoplastic or metal that is less compressible. In such an implementation, the sealing is primarily accomplished by the compression of a portion of the rotor 116 rather than the stator 114. In still other implementation, neither the rotor 116 nor the stator 114 are formed from elastomers and sealing is not achieved during the device operation. For example, a clearance gap may exist between the rotor 116 and stator 114 which creates unsealed cavities but when the rotor 116 is spinning the pumping assembly 110 still behaves like a pump but includes some slippage due to the clearance gap. The slippage may beneficially prevent efficient evacuation of low-viscosity fluids like blood but still enable pumping of thicker materials such as clot. This beneficially may reduce blood loss during clot extraction. Such clearance gaps are sometimes implemented in progressive cavity pumps to allow for manufacturing tolerances of the components.

In some implementations, the stator 114 has a distal edge that is rounded or tapered rather than sharp as shown in many of the figures. The rounded distal edge can prevent vessel injury by providing a smoother surface to contact the vessel wall.

In some implementations, the pumping assembly 110 does not pump fluid. For example, the stator helical lumen 210 or the rotor helical section 214 can be comprised of only a fraction of a full pitch cavity such as one-half. In such an implementation, a complete closed cavity 224 is not achieved between the stator helical lumen 210 and the rotor helical section 214 since there will be segments of the rotor 116 rotation where the lumen of the catheter body 104 is fluidly connected to the fluid at the distal end of the device 102. A separate aspiration source can be applied to the device 102, for example through the outlet tube 132, to draw fluid and material into the catheter body 104. The rotor 116 can then be rotated to assist in ingesting material that otherwise may have been clogged. In this manner, the tissue engagement portion 108 which includes the spinning rotor 116 works in conjunction with a separate aspiration source, which may include a pump, to remove fluid and material. The aspiration source may be a pump within the handle assembly 106 such as a piston pump, a peristaltic pump, or any other type of pump. The rotor 116 assists the aspiration power by disrupting the clot and urging pieces of the clot into the catheter body 104. In some implementations, the device 102 may not include a stator 114 and the rotor 116 may instead spin within the catheter body 104.

In some implementations, the device 102 may include more than one pumping assembly 110. For example, the catheter body 104 can have one or more pumping assemblies 110 along its length that further assist in removing the material from the patient or preventing the catheter body 104 from becoming clogged. The multiple pumping assemblies 110 can be rotationally connected by one or more torque members 122 like a daisy chain. In other implementations, the pumping assemblies 110 can be rotated at different speeds and timings. The pumping assemblies 110 can all have the same pumping parameters such as pitch and diameter or can be unique. In some implementations, the entire length of the catheter body 104 can have a single or multiple pumping assemblies 110. In addition, multiple inlet or outlet holes can exist on the catheter body 104. Some pumping assemblies 110 can be positioned within the body of the patient while others may be positioned outside the body of the patient during their intended use.

In some implementations, the distal bend 206 can be configured in various manners. In some implementations, the distal bend can be a rigid angle whereas in other configurations it can be flexible and deformable by the tissue structures or by the user prior to the operation. In other implementations, the distal bend 206 can be steerable. For example, the handle assembly 106 can include an interface that allows the user to actively adjust the angle of the distal bend 206. This can be accomplished with control wires or any other suitable mechanism. In some implementations, the rotation of the catheter hub 124 and the catheter body 104 can be done automatically by the handle assembly 106. For example, a separate motor can drive the rotation of the catheter hub 124 at a desired speed or in relation to the primary motor 416 which drives the pump assembly. In still other implementations, the rotation of the catheter hub 124 can be driven by motor 416, possibly through a gear reduction that spins the tip of the device one time for a given number of rotor 116 revolutions. The user may be able to provide input and control of the motorized rotation of the catheter hub 124. The spinning of the catheter hub 124 can help ensure that the distal bend 206 directs the tissue engagement portion 108 around the lumen of the blood vessel 604.

In some implementations, the controller 136 can apply different rotation profiles to the drive system 130 and motor 416. For example, in one implementation of a rotation profile, the motor speed is proportional to the throttle 418 user input. As the user depresses the throttle 418 further, the motor 416 spins faster. In other implementations of the rotation profile, the throttle 418 can toggle between certain discrete motor 416 speeds. For example, a low motor 416 speed may be useful for ingesting low flow rates of blood and acute soft clot 602. Feedback may be then provided to the user as described elsewhere that indicates that thicker clot 602 is encountered and therefore the user can depress the throttle 418 further to reach a higher discrete speed that is optimized for clot 602 ingestion. In other implementations of the rotation profile, the controller 136 can include additional profiles such as pulsed rotations where the pump is turned at high-speeds for discrete amounts of time with pauses in between. For example, the controller 136 can be configured to spin the motor 416 for a first discrete number of rotations at a first speed and then spin a second discrete number of rotations at a second discrete speed or alternatively may be configured to stop rotating for a discrete amount of time. The controller 136 can perform this pattern repeatedly. In still other implementations of the rotation profile, the controller 136 motor profile may include cycles of high-speed pumping with short bursts of rotation in the opposite direction that discharge small amounts of fluid. These profiles may improve clot disruption and ingestion while mixing the clot and blood. In some implementations, pulsed profiles can create a pulsed vacuum at the distal end of the catheter which can assist in ingesting clot. In some implementations of the rotation profile, the user can provide input into the controller 136 and adjust the rotation profile. Any number of motor rotation profiles may be contemplated.

In some implementations, the handle assembly 106 can be split between a disposable and a reusable assembly. For example, the catheter body 104 and catheter hub 124 along with all the distal elements and collection assembly 112 can be disposable while the majority of the handle assembly 106 can be a durable assembly. In this manner, the user can connect the disposable and reusable portions, perform the operation, and then dispose of the catheter assembly while cleaning and reusing the handle assembly 106. Any number of other configurations are contemplated.

In some implementations, the controller 136 can adjust the speed of motor 416 depending on the torque required to turn the rotor 116. For example, when the pumping assembly 110 ingests fluid, it will likely require a lower torque to turn than when the tissue engagement portion 108 is ingesting viscous material such as clot. One estimate of the torque applied is the current that the motor 416 uses to turn at a given speed. The controller 136 can define different profiles depending on the motor 416 current, where the profile provides a relationship between torque and rotational speed. So, as torque applied by the motor 416 varies, the controller 136 may adjust the rotational speed of the rotor 116 based on the profile.

For example, if the motor 416 is only ingesting blood it can turn at a slower speed and therefore reduce blood loss. In some implementations, a baseline torque can be individually set for each device by turning the motor in air, water, saline, or blood. For example, at manufacturing, the baseline torque can be saved in the controller 136 or alternatively can be established during an initialization or priming step performed by the user. This baseline can therefore be used for determining if the pumping assembly 110 is ingesting clot or engaged with other vascular structures. The motor 416 speed can be adjusted accordingly. In still other implementations, the baseline can be dynamically established by the controller based on a sampling of previously recorded measurements such as the motor current. Other methods of measuring the torque required to turn the rotor are contemplated such as motor voltage, torque gages, or any other suitable feedback. Once the tissue engagement portion 108 begins engaging with and ingesting clot, the motor 416 current increases and the controller 136 can increase the speed of the motor 416 to ingest the clot more efficiently. By way of example, the baseline torque can be established prior to beginning the procedure can be from and including about 0.1 mN·m to and including about 200 mN·m, or about 10 mN·m. The controller 136 can then determine that clot is engaged and change the speed of the motor 416 if the torque increases from and including about 5% to and including about 80%, or about 20%. Any number of profiles are contemplated in various states. In another implementation, the same method may be used to determine if a vessel or other fragile structure is engaged with the tissue engagement portion 108 and the controller 136 can stop the drive system 130 completely or even reverse it. Feedback can be provided to the user in any number of ways. For example, an auditory tone can be adjusted in volume or frequency and alert the user of whether the pumping assembly 110 is ingesting blood versus the tissue engagement portion 108 is encountering clot. Any number of user interfaces may be used such as light, sound, or vibration.

In some implementations, the device 102 can be connected to user interfaces such as smartphones, tablets, computers, or controller boxes. The connection with the device 102 can be made using Bluetooth, Wi-Fi, Zigbee, a wired connection, or any other suitable methods. The user can make adjustments to the controller 136 through the user interface and thereby change how the device 102 responds to different inputs such as the throttle 418. In some implementations, the device 102 can be controlled by other user inputs. For example, a foot pedal may be used as a throttle 418 rather than trigger.

In some implementations, the device 102 can record data from a given procedure and store the data in the controller 136 or transmit the data to a separate recording device via wireless or wired connection. The data can include information such as removed blood volume, torque profiles indicating clot engagement, duration of usage, or any other information generated during the procedure. The user can review the data or store it for future reference.

In some implementations, the filter assembly 712 can be located within the handle assembly 106. In such implementations, the expelled blood from the manifold 128 can be fed directly into a filter assembly 712 that separates clot material from the blood within the handle assembly 106. The return tube 716 can feed the filtered blood through the catheter body 104 and exit a side hole. In this manner, the device 102 may not require an external tube which may simplify set-up and operation. Filtered clot can be contained within a collection assembly 112 within the handle assembly 106. In some implementations, the catheter body 104 can include a multitude of fenestrations proximal to the pumping assembly 110 that allow blood to return to the patient but filter the clot and keep it within the catheter body 104. In this implementation, the catheter body 104 itself or a component within the catheter body 104 can act as a filter assembly 712.

In some implementations, the pump assembly may be run in reverse to what is described in FIGS. 3A-3F to dispense materials out of the pumping assembly 110. For example, if the pumping assembly 110 or tissue engagement portion 108 becomes clogged, the controller 136 can turn the drive system 130 counter-clockwise and therefore dispense material out of the pumping assembly 110. This can beneficially unclog the pumping assembly 110 and allow the user to resume the removal of the material. In some implementations, this can be done at fixed intervals of run time or at the end of a pumping operation to automatically unclog the device without input from the user. In other implementations, the reversal of the pumping assembly 110 can be used to let go of vessels or valves or other tissue which is not intended to be engaged by the tissue engagement portion 108.

In some implementations, the stiffness of the stator 114 material can be selected based on the material to be removed. For example, when removing acute fresh clot, the user may select a device 102 which has a stator 114 comprised of a more elastic material such as a silicone Shore 35A. When removing thicker chronic clot, the user may select a device 102 with a stator 114 comprised of nylon or a similarly rigid plastic.

In some implementations, the device 102 may be primed with fluid before use. For example, the user may place the tip of the device 102 into a container of saline and activate the device 102 thereby pumping saline into the catheter body 104. Alternatively, the user may prime the pumping assembly 110 with a grease or lubricant prior to clinical use.

### Experimental Results

A prototype device was built according to the implementation and design shown in FIG. 1A. The stator 114 was comprised of an elastic Shore 80A material and other materials were 3D printed in hard plastic. The catheter body 104 was an HDPE extrusion and a 24-volt brushless DC motor 416 was used in the drive system 130. A test was performed to evaluate the ability to extract clot from a simulated blood vessel. An artificial clot simulant weighing 6.3 gm was placed between two clear plates which included a cylindrical cutout. The model was pressurized with saline from a gravity fed reservoir at approximately 12 cm high. The device 102 was inserted through a hemostasis valve and into the model. The pumping assembly 110 was activated by the throttle 418 and motor 416. As the rotor 116 turned, fluid pumped through the device and out of the outlet tube. The tissue engagement portion 108 was then advanced toward the clot simulant. The tissue engagement portion 108 and pumping assembly 110 efficiently removed the clot simulant in 19 seconds and the measured volume of expelled fluid was only 52 ml. The experimental results demonstrate that this implementation of the device 102 performed as intended.

Much description has been given to thrombectomy procedures where clot and thrombus is removed but that is not intended to limit the scope or use of the device 102 to such procedures. For the removal of doubt, the terms clot and thrombus can be considered interchangeable with any material that is being removed. The device 102 can have a variety of shapes and sizes serving as a platform for any type of thrombectomy, embolectomy, or foreign body, calculi, or tissue removal in any part of the body or vessel. This could include but not limited to cerebral thrombi causing ischemic strokes, deep venous thrombosis both acute and chronic, pulmonary emboli, dural sinus thrombosis, controlled aspiration of tissue and/or fluid during surgery of the ventricular system or cerebrum, removal of liquid embolic agent, clotted hemodialysis grafts, peripheral arterial thromboemboli, including the mesenteric and peripheral vascular tree, peripheral arterial occlusion, critical limb ischemia (CLI), chronic total occlusion (CTO) and stone removal. The device 102 may also be used for debulking procedures for the removal of tumor and other cancerous materials.

Any number of other suitable applications may use such a device 102 for the removal of a tissue, foreign body, calculi, or other objects within a tubular contained space or even within non-tubular or non-contained spaces. In some implementations, the device 102 may be used for removal of tissue during small port laparoscopic procedures include biopsies or removal of malignant tissue.

The names and labels applied to the various components and parts should not be considered limiting to the scope of the invented device and method.

Although implementations of various methods and devices are described herein in detail with reference to certain versions, it should be appreciated that other versions, implementations, methods of use, and combinations thereof are also possible. Therefore, the spirit and scope of the appended claims should not be limited to the description of the implementations contained herein.

### Illustrative Aspects of the Disclosure

Illustrative examples of the disclosure include, but are not limited to the following:

Aspect 1: A device, including a catheter including a lumen extending through the catheter; and a progressive cavity pump at a distal portion of the catheter, wherein the progressive cavity pump is in communication with each of the lumen and an external environment surrounding the distal portion of the catheter and wherein the progressive cavity pump is operable to transfer material from the external environment into the lumen of the catheter.

Aspect 2: The device of Aspect 1 further including a container in fluid communication with a proximal end of the lumen, wherein the container is configured to provide or receive material transferred through the lumen.

Aspect 3: The device of Aspect 1 wherein the progressive cavity pump is coupled to and extends from a distal end of the catheter.

Aspect 4: The device of Aspect 1, wherein the progressive cavity pump is at least partially disposed within the lumen at a distal end of the catheter.

Aspect 5: The device of Aspect 1, wherein the progressive cavity pump includes a stator at least partially formed from an elastomer.

Aspect 6: The device of Aspect 1, wherein the progressive cavity pump includes each of a stator defining a helical stator lumen and a helical rotor extending through the helical stator lumen, and wherein a pitch of the helical stator lumen is about twice a pitch of the helical rotor.

Aspect 7: The device of Aspect 1, wherein the progressive cavity pump includes a stator and a rotor arranged to form a cavity between the stator and the rotor, and wherein the cavity is fluidically sealed due to compression between the stator and the rotor.

Aspect 8: The device of Aspect 1, wherein the progressive cavity pump includes a stator and a rotor arranged to form a cavity between the stator and the rotor, and wherein the cavity is unsealed due to a clearance gap between the stator and the rotor.

Aspect 9: The device of Aspect 1, wherein the progressive cavity pump includes a rotor, the device further including a torque member extending through the lumen and coupled to the rotor, the torque member operable to transfer a torque applied to a proximal end of the torque member to the rotor.

Aspect 10: The device of Aspect 1, wherein the progressive cavity pump includes a rotor, the device further including a motor coupled to the rotor and operable to drive the rotor.

Aspect 11: The device of Aspect 1, wherein the distal end of the catheter includes an angled tip.

Aspect 12: The device of Aspect 1, wherein the distal portion has an outer diameter from and including about 6 French to and including about 20 French.

Aspect 13: The device of Aspect 1, wherein the progressive cavity pump forms a seal between the lumen and the external environment.

Aspect 14: The device of Aspect 1 further including a handle assembly coupled to a proximal end of the catheter, wherein the handle assembly includes a drive system for controlling the progressive cavity pump.

Aspect 15: The device of Aspect 1 further including a distal pumping assembly coupled to a distal end of the catheter and including the progressive cavity pump, wherein the distal pumping assembly includes a sleeve that is coupled to and rotationally fixes a stator of the progressive cavity pump relative to the sleeve.

Aspect 16: The device of Aspect 1 further including a distal pumping assembly disposed within a distal end of the catheter and including the progressive cavity pump.

Aspect 17: The device of Aspect 1 further including a distal pumping assembly coupled to a distal end of the catheter and including the progressive cavity pump, wherein the progressive cavity pump includes each of a stator and a rotor, the distal pumping assembly includes a locating element disposed at a proximal end of the stator, and the locating element is configured to maintain an axial relationship between the stator and the rotor during operation of the distal pumping assembly.

Aspect 18: The device of Aspect 1, wherein the progressive cavity pump includes a rotor coupled to a drive system, the drive system configured to rotate the rotor at a plurality of rotational speeds.

Aspect 19: The device of Aspect 18, wherein the drive system is configured to change between speeds of the plurality of rotational speeds in response to a torque applied by the drive system.

Aspect 20: The device of Aspect 19, wherein the drive system the torque applied by the drive system is estimated based on a current drawn by a motor of the drive system.

Aspect 21: The device of Aspect 19, wherein the drive system includes a profile defining one or more relationships between torque and rotational speed, and wherein the drive system is configured to change between speeds of the plurality of rotational speeds in response to the torque based on the profile.

Aspect 22: The device of Aspect 1 further comprising a collection assembly in communication with the lumen of the catheter configured to receive material ingested by the progressive cavity pump, wherein the collection assembly includes a filtration system.

Aspect 23: The device of Aspect 22 further comprising a return tube in communication with the collection assembly configured to receive material not retained by the filter.

Aspect 24: The device of Aspect 1, wherein the progressive cavity pump includes a stator defining a stator lumen and a stator opening separate from the stator lumen, the device further comprising a visualization assembly supported within the stator opening.

Aspect 25: The device of Aspect 24, wherein the visualization assembly includes a cable and the stator defines a channel through which the cable extends.

Aspect 26: The device of Aspect 25, wherein the channel extends helically about the stator.

Aspect 27: The device of Aspect 1, wherein the progressive cavity pump includes a stator defining a stator lumen and a channel separate from the stator lumen.

Aspect 28: The device of Aspect 1, wherein the progressive cavity pump includes a stator defining a stator lumen and a channel separate from the stator lumen and extending along an external surface of the stator.

Aspect 29: The device of Aspect 1, wherein the progressive cavity pump includes a stator defining a stator lumen and a channel separate from the stator lumen and extending helically along an external surface of the stator.

Aspect 30: The device of Aspect 1, wherein the progressive cavity pump includes a stator defining a stator lumen and a plurality of channels separate from the stator lumen.

Aspect 31: The device of Aspect 1 further comprising a port extending between an internal volume of the device and the external environment and positioned proximal to the progressive cavity pump.

Aspect 32: The device of Aspect 31, wherein the port is sized to receive at least one of a guidewire and a microcatheter.

Aspect 33: A method including: locating a material removal device within a physiological lumen or cavity of a patient, the material removal device including: a catheter including a catheter lumen extending through the catheter; and a progressive cavity pump at the distal portion of the catheter, wherein the progressive cavity pump is in communication with each of the catheter lumen and the physiological lumen or cavity of the patient; actuating the progressive cavity pump to ingest material from the physiological lumen or cavity into the catheter lumen.

Aspect 34: The method of Aspect 33, further including transporting the material through the catheter lumen to a container disposed external to the patient and in communication with a proximal end of the catheter lumen.

Aspect 35: The method of Aspect 33, wherein the progressive cavity pump includes a stator and a rotor arranged to form a cavity between the stator and the rotor, wherein actuating the progressive cavity pump ingests the material into the cavity.

Aspect 35: The method of Aspect 33, wherein the material removal device is the device of any of Aspect 1 to 32.

Aspect 36: The method of Aspect 33, wherein the material removal device is the device of Aspect 18, and the method further includes rotating the rotor at a first speed of the plurality of rotational speeds; obtaining a torque applied to the rotor by the drive system while rotating the rotor at the first speed; and changing the rotational speed of the rotor to a second rotational speed of the plurality of rotational speeds in response to the torque.

Aspect 36: The method of Aspect 33, wherein the material removal device is the device of Aspect 19, and the method further includes rotating the rotor at a first speed of the plurality of rotational speeds; obtaining a torque applied to the rotor by the drive system while rotating the rotor at the first speed; and changing the rotational speed of the rotor to a second rotational speed of the plurality of rotational speeds in response to the torque and according to the profile.

Aspect 37: The method of Aspect 33 further including transferring the material to a collection assembly in communication with the catheter lumen and external the patient.

Aspect 38: The method of Aspect 37 further including filtering the ingested material using a filter assembly disposed within or in line with the collection assembly.

Aspect 39: The method of Aspect 37 further including returning at least a portion of the ingested material to the patient through a return tube in communication with the collection assembly.

Aspect 40: The method of Aspect 33, wherein the progressive cavity pump includes a stator defining a stator lumen and a channel separate from the stator lumen, the method further including at least one of infusing a substance or applying suction through the channel.

Aspect 41: The method of Aspect 33, wherein locating the material removal device includes locating the material removal device within a second catheter disposed within the physiological lumen or cavity of the patient.

Aspect 42: The method of Aspect 41, wherein the material is disposed within a lumen of the second catheter and ingesting material from the physiological lumen or cavity into the catheter lumen includes ingesting material from within the lumen of the second catheter.

Aspect 43: The method of Aspect 33, wherein locating the material removal device includes translating the material removal device along a guidewire.

Aspect 44: The method of Aspect 43, wherein the material removal device is the device of Aspect 31 and the guidewire extends through the port during translation of the material removal device along the guidewire.

What is claimed is:

1. A method of removing an occlusive material within a blood lumen of a patient, said method comprising:
positioning a material-removal device proximate to or in contact with said occlusive material within said blood lumen, said material-removal device comprising:
a catheter comprising a catheter lumen and a progressive cavity pump located at a distal portion of said catheter, said progressive cavity pump in fluid communication with said catheter lumen and said blood lumen; and
actuating said progressive cavity pump to ingest at least a portion of said occlusive material from said blood lumen into said catheter lumen.

2. The method of claim 1, wherein said occlusive material partially or completely occludes the blood lumen, and further wherein the blood lumen comprises at least one of a blood vessel and an artificial vascular graft.

3. The method of claim 1, wherein said occlusive material comprises at least one of clotted blood, thrombus and plaque.

4. The method of claim 1, further comprising depositing said at least a portion of said occlusive material from a proximal portion of said catheter lumen into a container located externally to the patient.

5. The method of claim 1, wherein said progressive cavity pump comprises a stator and a rotor, wherein said actuating the progressive cavity pump comprises forming at least one cavity between the stator and the rotor such that (i) when said at least one cavity is distally open, said at least a portion of said occlusive material is ingested into the at least one cavity, (ii) when said at least one cavity is closed, said at least a portion of said occlusive material is then translated proximally through said progressive cavity pump, and (iii) when said at least one cavity re-opens proximally, said at least a portion of said occlusive material is transferred from said progressive cavity pump into said catheter lumen.

6. The method of claim 5, wherein said stator comprises a first helical portion and said rotor comprises a second helical portion, and wherein said actuating the progressive cavity pump comprises rotating the second helical portion within said first helical portion so as to open and close said at least one cavity.

7. The method of claim 6, wherein said actuating the progressive cavity pump comprises (i) rotating the second helical portion within said first helical portion so as to form a plurality of cavities within said progressive cavity pump, and (ii) ingesting a plurality of portions of said occlusive material into said plurality of cavities within the progressive cavity pump.

8. The method of claim 6, wherein a first pitch of said first helical portion of said stator is about twice a second pitch of said second helical portion of said rotor.

9. The method of claim 5, wherein said at least one cavity, when closed, is fluidically sealed.

10. The method of claim 5, wherein said at least one cavity, when closed, remains fluidically unsealed.

11. The method of claim 1, wherein said progressive cavity pump is located either partially or entirely within, or is operatively coupled to, said distal portion of said catheter.

12. The method of claim 1 wherein the progressive cavity pump is operatively coupled to and extends distally from said distal portion of said catheter.

13. The method of claim 1, wherein said distal portion of said catheter comprises an outer diameter from and including about 6 French to and including about 20 French.

14. The method of claim 1, wherein said material-removal device further comprising a handle assembly coupled to a proximal end of the catheter, wherein said handle assembly comprises a drive system for controlling the progressive cavity pump.

15. The method of claim 1, wherein said material-removal device further comprises a torque member operatively coupled to said progressive cavity pump, and wherein said actuating said progressive cavity pump comprises rotating said torque member to rotate a rotor portion of said progressive cavity pump.

16. The method of claim 15, wherein said rotating said torque member comprising rotating said torque member either manually by a user or by a user actuating a motor operatively coupled to said torque member.

17. The method of claim 15, wherein said material-removal device further comprises a motor operatively coupled to said torque member for rotating said torque member to actuate said progressive cavity pump, said method further comprising varying a speed of rotation of said torque member during said actuating of said progressive cavity pump.

18. The method of claim 1, said method further comprising infusing at least one of saline, contrast and a therapeutic agent into the blood lumen during said method of treating said occlusive material.

19. The method of claim 1, where said progressive cavity pump is positioned or is positionable at an angle relative to a longitudinal axis of said catheter lumen, and wherein said actuating said progressive cavity pump further comprises rotating said progressive cavity pump within said blood lumen to treat a second portion of said occlusive material in said blood lumen.

20. A method of treating an occlusive material within a blood lumen of a patient, said method comprising:
positioning a material-removal device proximate to or in contact with said occlusive material within said blood lumen, said material-removal device comprising:
a catheter comprising a catheter lumen, and a pump located at a distal portion of said catheter, said pump in fluid communication with said catheter lumen and said blood lumen, wherein said pump comprises a stator comprising a first helical portion and a rotor comprising a second helical portion, and wherein rotation of said second helical portion of said rotor within said first helical portion of said rotor opens and closes at least one cavity formed between said stator and said rotor within said pump, and
actuating said pump to open and close said at least one cavity to ingest at least a portion of said occlusive material from the blood lumen into said at least one cavity and move said at least a portion of said occlusive material through the pump and into the catheter lumen.
